(19) United States
(12) Patent Application Publication
(10) Pub. No.: US 2026/0072265 A1
(43) Pub. Date: Mar. 12, 2026

(54) SYSTEMS AND METHODS FOR MULTIPLEX IMAGING

(71) Applicant: The Children's Medical Center Corporation, Boston, MA (US)

(72) Inventors: Jay R. Thiagarajah, Boston, MA (US); Michael D. Anderson, Boston, MA (US)

(73) Assignee: The Children's Medical Center Corporation, Boston, MA (US)

(21) Appl. No.: 19/388,358

(22) Filed: Nov. 13, 2025

Related U.S. Application Data
(63) Continuation of application No. PCT/US2024/031703, filed on May 30, 2024.
(60) Provisional application No. 63/505,322, filed on May 31, 2023.
(60) Provisional application No. 63/508,362, filed on Jun. 15, 2023.

(51) Int. Cl. G02B 21/36; B01L 3/00; G01N 21/64; G01N 33/58; G02B 21/26

(52) U.S. Cl. CPC G02B 21/365; B01L 3/502715; G01N 21/6428; G01N 21/6458; G01N 33/582; G02B 21/26; G02B 21/361; B01L 2200/027; B01L 2300/0654; B01L 2300/12; B01L 2400/043; B01L 2400/0487; B01L 2400/0655; G01N 2021/6439; G01N 2201/021

(57) ABSTRACT

The disclosure features systems and methods for multiplex imaging. In particular, improved systems and methods for multiplex imaging which are faster and more efficient than those known in the art are disclosed herein.

### CROSS-REFERENCE TO RELATED APPLICATIONS

The present application is a continuation under 35 U.S.C. § 111(a) of PCT International Patent Application No. PCT/US2024/031703, filed May 30, 2024, designating the United States and published in English, which claims priority to and the benefit US Provisional Application Nos. 63/505,322, filed May 31, 2023 and 63/508,362, filed Jun. 15, 2023, each of which is incorporated herein by reference in its entirety.

### BACKGROUND OF THE DISCLOSURE

Describing the spatial context of how proteins and cells organize, interact and change is critical to our understanding of tissue and organ biology in health and disease. Developments in multiplexed antibody-based, fluorescence labelling methods are now providing unique insights into tissue microenvironments. However, many current methods have a variety of drawbacks that reduce their practical utilization. Accordingly, improved methods for multiplex imaging are urgently needed.

### SUMMARY OF THE DISCLOSURE

As described below, the present disclosure features systems and methods for multiplex imaging.

In an aspect, the present disclosure provides an optical system for multiplex image acquisition. The system includes:
a) a microscope including
at least one light source configured to emit light towards a sample present in a chamber disposed within a chamber body;
an upright water dipping objective lens or water immersion lens configured to receive the light;
and a digital camera, a photodiode, or an optical detector configured to extract light captured by the objective lens, thereby taking an image of a sample;
b) a microfluidic chamber including an optical window positioned between the sample and the lens of the objective, where the viewing window has approximately the same refractive index as the solution in which the water-dipping objective is immersed and the solution present in the microfluidic chamber in which the sample is immersed, where the microfluidic chamber includes at least one inlet in fluid communication with the chamber, and at least one outlet in fluid communication with the chamber, where the flow of liquid through the microfluidic chamber is controlled by a controller operably connected to the microfluidic chamber;
c) a motorized stage in which the microfluidic chamber is disposed and at least one controller operably connected to the motorized stage, where the controller is configured to position the stage with submicron precision in X and Y axes, where the position of the stage is controlled by a controller operably connected to the motorized stage.

In another aspect, the present disclosure provides a microfluidic system including:
a microfluidic chamber including:
   a chamber body, and
   a viewing window including a fluorinated ethylene propylene (FEP) film.

In another aspect, the present disclosure provides a microfluidic chamber including a viewing window including a fluorinated ethylene propylene (FEP) film, at least one inlet, and at least one outlet, the inlet and outlet in fluid communication with the microfluidic chamber, where the flow of liquid through the microfluidic chamber is controlled by a controller operably connected to the microfluidic chamber.

In another aspect, the present disclosure provides a microfluidic system including:
a widefield upright microscope having a high-resolution XY stage with a fluid-immersion chamber and a slide surface, the fluid-immersion chamber having a magnet-embedded slide;
a microfluidic chamber positioned on the magnet-embedded slide and having
   a chamber body made from a Polydimethylsiloxane (PDMS) material and embedded with iron oxide dust, and
   a viewing window made from fluorinated ethylene propylene (FEP) film, the viewing window further including a FluoroEtch chemical material in the FEP film to increase bonding between the FEP film and the PDMS material of the chamber body; and
a magnet positioned below the slide surface, the magnet causing a magnetic force acting on the iron oxide dust of the PDMS material.

In another aspect, the present disclosure provides a microfluidic system including:
a widefield upright microscope having a high-resolution XY stage with a slide surface;
a microfluidic chamber positioned on the slide surface and having
   a chamber body made from a Polydimethylsiloxane (PDMS) material and embedded with iron oxide dust, and
   a viewing window made from fluorinated ethylene propylene (FEP) film, the viewing window further including a FluoroEtch chemical material in the FEP film;
a micro-peristaltic pump configured to control flow of fluorophores within the microfluidic chamber;
a magnet positioned below the slide surface, the magnet causing a magnetic force acting on the iron oxide dust of the PDMS material,
and a composition, where the composition is a buffer, a capture molecule conjugated to a fluorophore, or a peroxy acid.

In another aspect, the present disclosure provides a method of inactivating fluorescence from a fluorophore, the method including: contacting a fluorophore with a peroxy acid.

In another aspect, the present disclosure provides a method of inactivating a fluorophore bound to a biological sample. The method involves:
a) contacting a biological sample with a capture molecule conjugated to a fluorphore, where the capture molecule specifically binds a target present in the biological sample; and
b) contacting the sample with a peroxy acid, thereby inactivating the fluorophore.

In another aspect, the present disclosure provides a multiplex method for detecting two or more target molecules in a sample. The method involves:
a) contacting a biological sample with a capture molecule conjugated to a first fluorophore, where the capture molecule specifically binds a target molecule within the sample;
b) exciting the first fluorophore with an appropriate wavelength of light and detecting the fluorescence;
c) inactivating the fluorescence by contacting the biological sample with a basic composition including a peroxy acid; and
d) repeating steps a-c with one or more capture molecules, each conjugated to a fluorophore.

In any of the above aspects, or embodiments thereof, the microscope is an epifluorescence, confocal, or light sheet microscope.

In any of the above aspects, or embodiments thereof, the microfluidic chamber is a plurality of microfluidic chambers.

In any of the above aspects, or embodiments thereof, the viewing window includes fluorinated ethylene propylene (FEP) film.

In any of the above aspects, or embodiments thereof, the microfluidic chamber is disposed within a motorized stage, the motorized stage including
a metal plate configured to hold a liquid, the plate including at least two magnets;
a chamber body including Fe₂O₃ embedded Polydimethylsiloxane (PDMS) configured to hold a fluorinated ethylene propylene (FEP) film;
a sample chamber including an optical window, where the sample chamber is configured to accept a biological sample, the sample chamber further including a microfluidic system including at least one inlet in fluid communication with the sample chamber and at least one outlet in fluid communication with the sample chamber;
a planar surface comprising anti-reflective material positioned below the sample chamber;
a planar surface including a thermosetting resin that provides insulation, thermal conductivity, and/or mechanical support to the sample chamber.

In any of the above aspects, or embodiments thereof, the system further includes a magnet positioned below the chamber body.

In any of the above aspects, or embodiments thereof, the chamber body is made from a Polydimethylsiloxane (PDMS) material and embedded with iron oxide dust.

In any of the above aspects, or embodiments thereof, the viewing window is installed in the chamber body while the PDMS is in a curing state, the viewing window further including a FluoroEtch chemical material in the FEP film to increase bonding between the FEP film and the PDMS material of the chamber body.

In any of the above aspects, or embodiments thereof, the magnet is configured to enhance bond strength in the PDMS material, the magnet causing a magnetic force acting on the iron oxide dust of the PDMS material.

In any of the above aspects, or embodiments thereof, the system further includes a widefield upright microscope.

In any of the above aspects, or embodiments thereof, the system further includes an aluminum breadboard on which the widefield upright microscope is mounted.

In any of the above aspects, or embodiments thereof, the microscope includes a high numerical aperture (NA) water dipping objective.

In any of the above aspects, or embodiments thereof, the high NA water dipping objective includes objectives greater than 1.0 NA.

In any of the above aspects, or embodiments thereof, the system further includes a control board that is communicatively coupled to the microscope, the control board being configured to control an open-source microscopy control program via which the upright microscope is operated.

In any of the above aspects, or embodiments thereof, the microscope includes a high-resolution XY stage with a fluid-immersion chamber and a slide surface.

In any of the above aspects, or embodiments thereof, the microfluidic chamber is positioned on a magnet-embedded slide in the fluid-immersion chamber.

In any of the above aspects, or embodiments thereof, the system further includes a heating element configured to control the temperature of the microfluidic chamber.

In any of the above aspects, or embodiments thereof, the microfluidic chamber includes a biological sample. In any of the above aspects, or embodiments thereof, the biological sample is a formalin-fixed paraffin embedded (FFPE) tissue slide.

In any of the above aspects, or embodiments thereof, the system further includes a micro-peristaltic pump configured to control, via a multipinch valve, flow of liquids within the microfluidic chamber.

In any of the above aspects, or embodiments thereof, the viewing window has a refractive index that is the same as the refractive index of the water. In any of the above aspects, or embodiments thereof, the viewing window includes a fluorinated ethylene propylene (FEP) film. the widefield upright microscope includes a high numerical aperture (NA) water dipping objective.

In any of the above aspects, or embodiments thereof, the slide surface has a heating element embedded thereof.

In any of the above aspects, or embodiments thereof, the slide surface has a formalin-fixed paraffin embedded (FFPE) tissue slide.

In any of the above aspects, or embodiments thereof, the system further includes a micro-peristaltic pump configured to control, via a multipinch valve, flow of fluorophores within the microfluidic chamber.

In any of the above aspects, or embodiments thereof, the FEP film has a refractive index that is the same as the refractive index of the water.

In any of the above aspects, or embodiments thereof, the peroxy acid is present in a basic solution.

In any of the above aspects, or embodiments thereof, the peroxy acid inactivates fluorescence at least 2-1,000 times faster than the rate at which hydrogen peroxide inactivates the fluorescence under substantially similar conditions. In any of the above aspects, or embodiments thereof, the peroxy acid inactivates fluorescence at least 10 times faster than the rate at which hydrogen peroxide inactivates the fluorescence under substantially similar conditions. In any of the above aspects, or embodiments thereof, the peroxy acid inactivates fluorescence at least 50 times faster than the rate at which hydrogen peroxide inactivates the fluorescence under substantially similar conditions. In any of the above aspects, or embodiments thereof, the peroxy acid inactivates fluorescence at least 100 times faster than the rate at which hydrogen peroxide inactivates the fluorescence under substantially similar conditions. In any of the above aspects, or embodiments thereof, the peroxy acid inactivates fluorescence at least 500 times faster than the rate at which hydrogen peroxide inactivates the fluorescence under substantially similar conditions. In any of the above aspects, or embodiments thereof, the peroxy acid inactivates fluorescence at least 1,000 times faster than the rate at which hydrogen peroxide inactivates the fluorescence under substantially similar conditions.

In any of the above aspects, or embodiments thereof, the peroxy acid inactivates fluorescence without producing significant amounts of oxygen or hydrogen gas, without producing detectable amounts of oxygen or hydrogen gas, and/or without producing amounts of oxygen or hydrogen gas that interferes with imaging.

In any of the above aspects, or embodiments thereof, the peroxy acid is present in a composition having a pH greater than about 8, 9, 10, or 11.

In any of the above aspects, or embodiments thereof, the capture molecule is a polypeptide or polynucleotide, where the capture molecule is an antibody or antigen-binding fragments; small molecule; biotin/streptavidin; probe; or aptamer.

In any of the above aspects, or embodiments thereof, the fluorophore is Alexa 488, Alexa 532, Alexa 555, or Alexa 647. In any of the above aspects, or embodiments thereof, the fluorophore is DyLight 750.

In any of the above aspects, or embodiments thereof, the contacting is for less than about 10 minutes or less than about 5 minutes. In any of the above aspects, or embodiments thereof, the contacting is for about 3 minutes.

In any of the above aspects, or embodiments thereof, the sample is a biological sample.

In any of the above aspects, or embodiments thereof, the biological sample is a biological cell or tissue. In any of the above aspects, or embodiments thereof, the tissue is a formalin-fixed paraffin embedded (FFPE) tissue section.

In any of the above aspects, or embodiments thereof, the method further includes illuminating the fluorophore and detecting fluorescence from the fluorophore prior to contacting the fluorophore with the peroxy acid.

In any of the above aspects, or embodiments thereof, the target molecule of the first capture molecule is the same or different from the target bound by a subsequent capture molecule.

In any of the above aspects, or embodiments thereof, the fluorophore conjugated to the first capture molecule is the same or different from the fluorphore conjugated to a subsequent capture molecule.

In any of the above aspects, or embodiments thereof, the peroxy acid is: peracetic acid; m-CPBA; magnesium monoperphthalate; Payne's reagent; trifluoroperacetic acid; 2,4-dinitroperbenzoic acid; Caro's acid; or potassium caroate.

Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “antibody” is meant an immunoglobulin polypeptide having immunogen binding ability. Antibodies are evoked or elicited in subjects (humans or other animals or mammals) following exposure to a specific antigen (immunogen). A subject capable of generating antibodies/immunoglobulins (i.e., an immune response) directed against a specific antigen/immunogen is said to be immunocompetent. Antibodies are characterized by reacting specifically with (e.g., binding to) an antigen or immunogen in some demonstrable way, antibody, and antigen/immunogen each being defined in terms of the other.

By “basic” is meant a pH of greater than about 7.0. In embodiments, a basic pH may be a pH of from about 7.2 to about 12 (e.g., 7.3, 7.4, 7.5, 7.6, 7.7, 7.8, 7.9, 8.0, 8.5, 9.0, 10.0, 11.0, 12.0).

“Biological sample” as used herein means a biological material isolated from a subject. Exemplary biological samples include any tissue, cell, fluid, or other material obtained from or derived from the subject. A biological sample may be an in vivo sample or an in vitro sample. Biological sample may be of prokaryotic origin or eukaryotic origin. For example, the biological sample may be originated from a biological subject such as a bacterium, a fungus, a protozoan, an insect, a fish, a bird, a reptile, a mammal (e.g., mouse, rat, cow, dog, donkey, guinea pig, or rabbit), or a primate (e.g., chimpanzee, or human). A biological sample may be obtained or derived from a biological subject by a variety of methods. For example, a biological sample may include tissues or cells isolated from mammals (e.g., humans), sections of a biological sample (e.g., sectional portion of an organ or tissue), or extracts from a biological sample (e.g., an antigen extracted from a body fluid such as blood, blood plasma, serum, or urine). Non-limiting examples of the biological sample include, cells, cell fragments, tissues, tissue sections, organs, or body fluids. The biological sample may be immobilized on a solid support, such as in blots or arrays. For example, the biological sample may be immobilized on a membrane, a paper, a glass slide, a microtiter plate, or an ELISA plate.

In this disclosure, “comprises,” “comprising,” “containing”, and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “Capture Molecule” is meant a polypeptide or polynucleotide that specifically binds a target. Exemplary capture molecules include antibodies and antigen-binding fragments, small molecules, biotin/streptavidin, probes, and aptamers.

“Detect” refers to identifying the presence, absence or amount of the object to be detected.

By fluorophore” is meant a chemical compound, which when excited by exposure to a particular wavelength of light, emits light at a specific wavelength. Fluorophores may be described in terms of their emission profile, or “color.” Green fluorophores (for example Cy3, FITC, and Oregon Green) may be characterized by their emission at wavelengths generally in the range of 515-540 nanometers. Red fluorophores (for example Texas Red Cy5, and tetramethylrhodamine) may be characterized by their emission at wavelengths generally in the range of 590-690 nanometers. Examples of fluorophores include, but are not limited to, 4-acetamido-4′-isothiocyanatostilbene-2,2′disulfonic acid, acridine, derivatives of acridine and acridine isothiocyanate, 5-(2-aminoethyl)aminonaphthalene-1-sulfonic acid (EDANS), 4-amino-N-3-vinylsulfonyl)phenylnaphthalimide-3.5 disulfonate (LuciferYellow VS), N-(4-anilino-1-naphthyl)male imide, anthranilamide, Brilliant Yellow, coumarin, coumarin derivatives, 7-amino-4-methylcoumarin (AMC, Coumarin 120), 7-amino-trifluoromethylcouluarin (Coumaran 151), cyanosine; 4,6-diaminidino-2-phenylindole (DAPI), 5′5″-dibromopyrogallol-sulfonephthalein (Bromopyrogallol Red), 7-diethylamino-3-(4-isothiocyanatophenyl)4-methyl coumarin, 4,4′-diisothiocyanatodihydro-stilbene-2,2′-disulfonic acid, 4,4′-diisothiocyanatostilbene-2,2′-disulfonic acid, 5-dimethylaminonaphthalene-1-sulfonyl chloride (DNS, dansyl chloride), eosin, derivatives of eosin, such as eosin isothiocyanate, erythrosine, derivatives of erythrosine, such as erythrosine B and erythrosin isothiocyanate; ethidium; fluorescein and derivatives such as 5-carboxyfluorescein (FAM), 5-(4,6-dichlorotriazin-2-yl)aminofluorescein (DTAF), 27-dimethoxy-4′5′-dichloro-6-carboxyfluorescein (JOE), fluorescein, fluorescein isothiocyanate (FITC), QFITC (XRITC); fluorescamine derivative (fluorescent upon reaction with amines); IR 144; IR 1446; Malachite Green isothiocyanate; 4-methylumbelliferone; ortho cresolphthalein; nitrotyrosine; pararosaniline; Phenol Red, B-phyco erythrin; o-phthaldialdehyde derivative (fluorescent upon reaction with amines); pyrene and derivatives such as pyrene, pyrene butyrate and Succinimidyl 1-pyrene butyrate; Reactive Red 4 (Cibacron® Brilliant Red 3B-A), rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl Rhodamine, tetramethyl rhodamine isothiocyanate (TRITC); riboflavin; rosolic acid and lathanide chelate derivatives, quantum dots, cyanines, pyrelium dyes, and squaraines. In some embodiments, the fluorophore comprises at least one alkene group (C═C) capable of forming an epoxide with a peroxy acid. In some embodiments, the fluorophore is a member of the Alexa Fluor™ family of fluorophores (e.g., Alexa 488, 532, 555, or 647). In one embodiment, the fluorophore is DyLight™ 750.

The term “inactivating,” as used herein, refers to reducing fluorescence of a fluorophore. In some embodiments, the inactivation results in a reduction in fluorescence by at least about 50%, 60%, 70%, 80%, 90% or more. In one embodiment, inactivation of at least about 90% of fluorescence is achieved. For example, if a plurality of fluorophores are activated to produce 10 MESF (molecules of equivalent soluble fluorochrome) of fluorescence, inactivating the plurality of fluorophores would reduce fluorescence to about 1 MESF. In some embodiments, after inactivation about 1, 2, 3, 4, 5, 6, 7, 8, 9, 10% of the original fluorescence is detected.

The term “in situ,” refers to an event occurring in the original or native location. For example, in situ may refer to an event occurring in an intact organ or tissue, or in a representative segment of an organ or tissue. In situ analysis of targets often provides contextual information that may be lost when the target is removed from its site of origin. In situ analysis of targets may be performed on cells, tissues, or tissue sections derived from a variety of sources, including an organism, an organ, a tissue, or a cell culture. The cell membrane may be fully intact or partially intact while performing the in situ analysis. Furthermore, the methods may be employed to analyze targets in situ in cell or tissue samples that are fixed, unfixed or frozen.

The term “observing a signal” refers to detecting, characterizing, or monitoring the signal, e.g., fluorescence. A signal from a fluorophore may be observed using a detection system or an imaging system. For example, observing a signal from a biological sample may be performed by capturing an image of the biological sample. Non-limiting examples of detection/imaging system that may be used to observe a signal include an optical system (e.g., optical imaging, fluorescence imaging, confocal imaging), an electrical system, a photographic film system, and a chemiluminescent system. Observing a signal also includes visual observation of a signal.

By “peroxy acid” (also known as a peroxyacid or a peracid) is meant an acid which includes an acidic —OOH group. Peroxy acids have the general formula shown below:

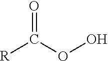

Exemplary peroxy acids include, without limitation: peracetic acid; m-CPBA; magnesium monoperphthalate; Payne's reagent; trifluoroperacetic acid; 2,4-dinitroperbenzoic acid; Caro's acid; and potassium caroate. In embodiments, the peroxy acid is m-CPBA.

The phrase “rate at least nX faster,” as used herein, refers to achieving inactivation within 1/n of the time it takes another composition (e.g., hydrogen peroxide) to achieve inactivation under same or similar conditions. For example, if a first composition requires 10 minutes to achieve inactivation, then a composition with a rate at least 5× faster is able to achieve inactivation in 2 minutes or less. In terms of the time constant (τ) for achieving inactivation, if a first composition is 5× faster than a second composition, then the τ of the first composition is a fifth of the τ of the second composition. In embodiments, n is at least 2-1,000. In embodiments, n is at least 2, 3, 4, 5, 6, 7, 8, 9, 10, 15, 20, 25, 30, 35, 40, 45, 50, 100, 150, 200, 250, 300, 350, 400, 450, 500, 550, 600, 650, 700, 750, 800, 850, 900, 950, or 1,000.

By “reference” is meant a standard or control condition.

By “refractive index” is meant the ratio between the speed of light in a medium to the speed of light in a vacuum. The refractive index (n) equals the velocity of light in a vacuum (c) divided by the velocity of light in a medium of interest (v):

n=c/v. In an embodiment, the refractive index of water is about 1.333.

The term “specific binding,” as used herein, refers to the specific recognition of one of two different molecules (partner molecules) for the other compared to substantially less recognition for other, non-partner molecule(s). Generally, specificity of a binding event results from specific molecular recognition sites in the partner molecules. The molecules may have recognition sites either on their surfaces or in their cavities that give rise to specific recognition between the two partner molecules. Molecular recognition may result via electrostatic interaction, hydrogen bonding, hydrophobic interaction, or a combination of interactions thereof. Examples for specific binding interactions include, but are not limited to, antibody-antigen interaction, enzyme-substrate interaction, complementary nucleic acid sequence hybridization, ligand-receptor interaction, and the like. Target binding ligands are selected in such a way that they bind specifically to their respective targets.

By “substantially similar conditions” is meant the same buffer, the same fluorophore, the same temperature, and/or the same pH. In some embodiments, the pH used is about 9 or 10.

The term “target” or “target molecule,” as used herein, generally refers to a polypeptide or polynucleotide that may be detected or analyzed when present in the biological sample. The target may be any substance for which there exists a naturally occurring target binding moiety (e.g., an antibody to a target antigen), or for which a target binding moiety may be prepared (e.g., a synthetic small molecule binder such as a ligand for a target receptor). Non-limiting examples of suitable targets include peptides, proteins, oligonucleotides, nucleic acids (e.g., DNA or RNA), polysaccharides (e.g., lectins or sugars), lipids, ligands, receptors, antibodies, affibodies, antigens, aptamers, haptens, hormones, enzymes, enzyme substrates, or combinations thereof.

The term “target binding ligand,” as used herein, refers to a molecule and/or moiety that is capable of specifically binding to a target. The target binding ligand may be used to detect or analyze a target when the target is present in the biological sample. The target binding ligand comprises at least one target binding moiety. The target binding moiety may be a naturally occurring specific binder of the target, a moiety that is derived from the naturally occurring specific binder, or a synthetic moiety that specifically binds to the target. For example, a target binding ligand to detect a target antigen may be derived from an antibody that is specific to the target antigen. The target binding ligand may further comprise a signal-generating moiety, a masked signal-generating moiety, a cross-linking moiety, or an independently detectable moiety associated (either covalently or non-covalently) with the target binding moiety. Often, the target binding ligands bind the target via molecular recognition of discrete chemical moieties of the target or structural components of the target (e.g., via molecular recognition of a specific three-dimensional structure of a protein). Suitable examples of target binding ligands include, but are not limited to, a DNA, a RNA, a locked nucleic acid (LNA), a peptide nucleic acid (PNA), a modified oligonucleotide (e.g., oligonucleotide with a modified base, a modified sugar moiety, or a modified phosphate moiety), an antibody, an antibody fragment, a peptide, an affibody, a hapten, a ligand, or an aptamer. In some preferred embodiments, the target binding ligand is an antibody or an antigen binding fragment thereof.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1F provide schematics, a flow diagram, plots, graphs, and images. (FIG. 1A) Schematic of overall SPECTRE-plex system design. (FIG. 1B) Overview of the process and cyclical steps and experimental times for SPECTRE-plex based tissue staining. (FIG. 1C) Kinetics of dye inactivation of Alexa 488, 532, 555, and 647) showing time constant (τ, s/mM) for meta-Chloroperoxybenzoic acid (m-CPBA) also termed peroxycarboxylic acid, LiBH4, and H₂O₂ (left) and time course of m-CPBA dye-inactivation for susceptible fluorophores Alexa 555 and 647 and non-susceptible Alexa 594 (right). In the left panel, the bars are ordered in each set of three, from left to right, as follows: m-CPBA; LiBH4; H2O2. In the right panel, the traces are ordered, from top to bottom, as follows: A594; A555; A647. (FIG. 1D) Schematic for imaging chamber system including microfluidic (‘invisi-slip’) device. (FIG. 1E) High magnification image of human duodenum using 60×, 1.2NA objective. (FIG. 1F) Antibody binding association of anti-MUC2 IgG on duodenal tissue with alteration of solution pH (top), and temperature (bottom) with example images of antibody fluorescence (right). In the bottom panel, the top line is 37° C. and the bottom line is 22° C.

FIGS. 2A-2F provide images, plots, and graphs. (FIG. 2A) Example tiled, stitched and processed whole section tissue images from healthy control duodenum and active celiac disease. Main image showing lactase, mucin-2 (MUC2), smooth muscle actin (SMactin), sodium-potassium ATPase (Na⁺K⁺-ATPase) and nuclei (DAPI), with inset target antigens as indicated. (FIG. 2B) Cell-type spatial map of healthy and celiac disease duodenum. (FIG. 2C) Uniform Manifold Approximation and Projection (UMAP) for spatial clustering of cell-types. (FIG. 2D) Mean cell numbers and cell percentages for healthy versus celiac disease tissue (n=2). (FIG. 2E) Spatial neighborhood linear distance cluster analysis for pEGFR+ tuft cells. (FIG. 2F) Spatial neighborhood density analysis for PCNA+ cycling epithelial cells.

FIGS. 3A-3B provide schematics showing the imaging chamber design. (FIG. 3A) 3D rendering of system with light path and lens superimposed. The elements in the light path are an (1) 38 mm focal length collimating, liquid light guide coupling lens, (2) excitation tube lens, (3) pentapass dichroic, (4) Nikon 16×0.8NA water dipping objective, (5) emission tube lens, (6) emission filters in an emission filter wheel and a photometric IRIS 15 camera. (FIG. 3B) 3D rendering of imaging chamber with slide into alignment jig, invisi-slip fluidic device on slide and pressure plate in place over invisi-slip device.

FIG. 4 provides flow diagrams showing a comparison of SPECTRE-Plex with manual or semi-automated tissue-based cyclic immunofluorescence (tCYCIF). Estimated total experimental times for a 7 cycle multiplex run based on manual and semi-automated tCYCIF protocols (left) versus the SPECTRE-Plex system (right) are provided.

FIGS. 5A-5B provide a reaction diagram and a graph showing the chemical reaction for the novel dye inactivation method of the present disclosure. (FIG. 5A) The proposed chemical reaction is epoxidation of the alkene groups via the Prizelhaev reaction followed by the formation of vicinal diols via the addition of NaOH. (FIG. 5B) Bleaching of Alexa-488 in the presence of 10 mM meta-chloroperoxybenzoic acid (m-CPBA) and 20 mM NaOH was analyzed using a kinetic model based on either single or double exponential fitting. m-CPBA is a more efficient catalyst of the Prizelhaev reaction in comparison to hydrogen peroxide. The kinetic data supported a sequential 2-part reaction. It was observed that cyanine-based and rhodamine-based fluorophores were susceptible to bleaching.

FIGS. 6A-6B provide an image and a plot showing a comparison of mCPBA over LiBH4 and H₂O₂ for dye inactivation and oxygen production. (FIG. 6A) Falcon tubes containing 10 mM mCPBA, 4.5% H₂O₂ and 1 mg/ml LiBH4 showing gas production over 20 minutes. (FIG. 6B) Comparison of τ values for bleaching.

FIG. 7 provides an image and a schematic showing the “invisi-slip” microfluidic device. Shown is the overall design of the “invisi-slip” microfluidic device.

FIGS. 8A-8B provide flow diagrams showing the image acquisition outline. (FIG. 8A) An overview of the hardware and software components and their interconnectedness is outlined. (FIG. 8B) A visual guide to the overall acquisition process.

FIG. 9 provides a flow diagram and a graph showing an overview of SPECTRE-plex autofocus routine using a optimized Brenner Score.

FIG. 10 provides an image showing an overview of the autoexposure routine.

FIG. 11 provides a flow diagram showing an overview of the image post-processing pipeline.

FIG. 12 provides a flow diagram showing a schematic of the image analysis pipeline.

FIG. 13 provides images showing cell nuclei segmentation when viewed using the multiplex system and methods of the present disclosure.

FIG. 14 is a schematic illustration of a microfluidic system, in accordance with aspects of the present disclosure.

FIG. 15 is a diagrammatic illustration of the microfluidic system of FIG. 14.

FIG. 16 is a perspective view of a microfluidic system in a laboratory setting, in accordance with other aspects of the present disclosure.

FIG. 17 is a perspective view of a microfluidic system, in accordance with other aspects of the present disclosure.

FIG. 18 is an exploded view of the microfluidic system of FIG. 17.

FIG. 19 is a partial cross-sectional illustration along line “6”-“6” of the microfluidic system of FIG. 17.

FIG. 20A is a perspective view of a microfluidic device, in accordance with yet other aspects of the present disclosure.

FIG. 20B is a top view diagrammatic illustrating components of the microfluidic device of FIG. 20A.

FIG. 20C is a side view diagrammatic illustrating steps in constructions of a viewing window of the microfluidic device of FIG. 20A.

FIG. 21 is a perspective view of a microfluidic chamber, in accordance with yet other aspects of the present disclosure.

FIG. 22 is a perspective view illustrating the microfluidic chamber of FIG. 21 with a microfluidic device.

FIG. 23 is a perspective illustrating the microfluidic chamber of FIG. 21 positioned on a stage.

FIG. 24 is a perspective view illustrating a fluidic system with a pump, in accordance with yet other aspects of the present disclosure.

FIG. 25 illustrates the fluidic system of FIG. 24 with a valve, in accordance with yet other aspects of the present disclosure.

FIG. 26 is a diagrammatic illustrating software and controllers of a microfluidic system, in accordance with yet other aspects of the present disclosure.

### DETAILED DESCRIPTION OF THE DISCLOSURE

The disclosure features systems and methods for multiplex imaging.

The disclosure is based, at least in part, on the discovery that optimized microscopy systems described herein are especially well suited for multiplex imaging. The disclosure is also based, at least in part, upon the discovery that peroxy acids (e.g., meta-chloroperoxybenzoic acid (m-CPBA)) are more efficient catalysts of the process of fluorescent dye inactivation/bleaching.

Describing the spatial context of how proteins and cells organize, interact and change is critical to the understanding of tissue and organ biology in health and disease. Several different high dimensionality spatial biology approaches for tissues have emerged primarily either based on detection and measurement of mRNA transcripts (e.g. MERFISH, Slide-seq) (Chen et al., Science 348, aaa6090 (2015); Rodrigues et al. Science 363, 1463-1467 (2019)) or protein (e.g CODEX, cyCIF) (Goltsev et al., Cell 174, 968-981.e15 (2018); Gerdes et al., Proceedings of the National Academy of Sciences 110, 11982-11987 (2013); Lin et al., Elife 7, e31657 (2018); Saka et al., Nat Biotechnol 37, 1080-1090 (2019); He et al., Nat Biotechnol 40, 1794-1806 (2022)).

Detection and localization of proteins across tissues allows assessment of cellular function, local communication, and accurate mapping of tissue state. Imaging-based protein methods center around cognate antibody binding to targets of interest, with either fluorescence or mass-spectroscopy-based detection. To detect multiple targets in the same tissue, most multiplexed protein techniques are based on cyclical, iterative antibody binding (Chen & Guo ACS Meas Sci Au 2, 296-303 (2022)). Distinction of individual targets between cycles can be achieved in a variety of ways such as fluorescence dye inactivation (t-cyCIF, IBEX), barcoding (CODEX) or antibody elution (Goltsev et al., Cell 174, 968-981.e15 (2018); Lin et al., Elife 7, e31657 (2018); Radtke et al., Proc Natl Acad Sci USA 117, 33455-33465 (2020); Zrazhevskiy & Gao, Nat Commun 4, 1619 (2013)). Each of the major methods has relative advantages and disadvantages varying by application, ease of use, and resource investment.

For example, while CODEX enables highly amplified and specific signal identification, the need for specific oligonucleotide conjugation substantially increases cost (for commercially available targets/panels) and/or time (for custom antibody conjugation and validation). Antibody elution/stripping methods have advantages in linear signal amplification but can be hampered by incomplete antibody removal and loss of tissue morphology after a finite number of cycles (Chen & Guo ACS Meas Sci Au 2, 296-303 (2022)). Dye inactivation methods based on directly conjugated fluorescent antibodies are attractive because of the large array of cheap, validated commercially available antibodies, and relatively simple procedures and reagents. However, current non-commercial and commercial versions of dye inactivation methods have drawbacks related to cost, speed, resolution, and fluidics.

### Multiplex Immunofluorescence Imaging

Multiplex immunofluorescence staining and imaging of human and animal tissues have become a major technique to spatially investigate proteins in health and disease states. Multiplex immunofluorescence imaging relies on multi-channel fluorescence microscopy imaging. These methods allow many proteins (e.g., 6-80) to be labeled and imaged on the same tissue section. Cyclical methods involving iterative labeling, and either antibody removal or fluorescence inactivation form the core of most methods.

Antibody removal or stripping can degrade tissues limiting the number of rounds of staining. Cyclical methods involving deoxyribonucleic acid (“DNA”) barcoding of antibodies, and secondary fluorescent oligonucleotides have the advantage of enabling large numbers of potential targets, but require purchasing of a relatively small array of existing bar-coded antibodies or custom barcoding of target antibodies. Thus, one of the problems with many of these cyclical methods is that they are expensive on a per slide basis.

Fluorescence inactivation methods also have several drawbacks. One drawback is that they require manual and tedious staining. Another drawback is that they require a lot of time to iterate through each stain and dye inactivation image cycle. Yet other drawbacks relate to difficulty in registration of images and the limited number of fluorophores that can be bleached. Another major drawback with current systems for repeated multiplex staining and imaging is the need for a glass coverslip or viewing window, which limits imaging resolution due to the requirement for the use of air objectives (and therefore a numerical aperture (“NA”) less than 0.9). The system and methods described herein address these drawbacks, and provide an end-to-end solution based on a series of methods that significantly improve speed, automation, and resolution of cyclic multiplex immunofluorescence imaging.

### Fluorescence Microscopy

Fluorescence microscopy is a staple component of life science and pharmaceutical research. Many areas of life science and pharmaceutical research rely heavily on fluorescence microscopy to observe biological samples at the sub-cellular level, allowing sub-cellular components to be imaged at high resolution. Orthogonal labelling strategies also enable different components to be imaged in the same cell, using multiple channels (e.g. based on the use of multiple fluorophores) and the composition of these multi-channel micrographs are extremely useful in a variety of downstream analyses.

The ability to image different channels from the same sample typically requires exploitation of known excitation/emission spectra from various fluorescent proteins or dyes. Fluorescence microscopy is based on the detection of fluorescent compounds, which in turn may be used in order to generate an image of a sample (which may be instead of, or in addition to the detection of light). Fluorophores are fluorescent chemical compounds or biological proteins that can re-emit light upon light excitation. In fluorescence microscopy fluorophores are illuminated with light of one or more specific wavelengths. This light is absorbed by the fluorophore(s), and the energy of the fluorophore is briefly raised to a higher excited state. The subsequent return to ground state results in emission of fluorescent light that can be detected and measured. The detection of the emitted light may be used to generate an image.

Fluorophores are thus molecules that emit light in response to light excitation, the re-emitted light being at a longer wavelength than the light used for excitation. Any given fluorophore will have a defined maximum excitation and emission wavelength which corresponds to the peak in the excitation and emission spectra. Examples include fluorescein (FITC), rhodamine derivatives (TRITC), coumarin derivatives and cyanine derivatives. Fluorescent microscopy may, for example be used to detect the presence or location within a sample of one or more target molecules. Certain target molecules, such as nucleic acids, can be detected using fluorescent stains (which may be small molecules which are intrinsically fluorescent). Nucleic acid stains such as DAPI and Hoechst are suitable examples, which bind the minor groove of DNA, and are therefore routinely used to fluorescently label cell nuclei. To detect the presence or location within a sample of other target molecules, the ability of specific binding agents to bind to target molecules can be manipulated. It is well known to use antibodies as binding agents, for example. Antibodies against a particular target molecule can be used in such methods. The primary antibody (which binds to the target molecule) may be labelled with a fluorescent molecule, or this fluorescent molecule may be carried on a second antibody that binds to the primary antibody. In this way, the detection of fluorescence at a particular location within the sample indicates that the target molecule is present at that location.

Fluorophores useful for fluorescence microscopy may be described in terms of their emission profile, or “color.” Green fluorophores (for example Cy3, FITC, and Oregon Green) may be characterized by their emission at wavelengths generally in the range of 515-540 nanometers. Red fluorophores (for example Texas Red Cy5, and tetramethylrhodamine) may be characterized by their emission at wavelengths generally in the range of 590-690 nanometers. Examples of fluorophores include, but are not limited to, 4-acetamido-4′-isothiocyanatostilbene-2,2′disulfonic acid, acridine, derivatives of acridine and acridine isothiocyanate, 5-(2-aminoethyl)aminonaphthalene-1-sulfonic acid (EDANS), 4-amino-N-3-vinylsulfonyl)phenylnaphthalimide-3.5 disulfonate (LuciferYellow VS), N-(4-anilino-1-naphthyl)male imide, anthranilamide, Brilliant Yellow, coumarin, coumarin derivatives, 7-amino-4-methylcoumarin (AMC, Coumarin 120), 7-amino-trifluoromethylcouluarin (Coumaran 151), cyanosine; 4,6-diaminidino-2-phenylindole (DAPI), 5′5″-dibromopyrogallol-sulfonephthalein (Bromopyrogallol Red), 7-diethylamino-3-(4-isothiocyanatophenyl)4-methyl coumarin, 4,4′-diisothiocyanatodihydro-stilbene-2,2′-disulfonic acid, 4,4′-diisothiocyanatostilbene-2,2′-disulfonic acid, 5-dimethylaminonaphthalene-1-sulfonyl chloride (DNS, dansyl chloride), eosin, derivatives of eosin, such as eosin isothiocyanate, erythrosine, derivatives of erythrosine, such as erythrosine B and erythrosin isothiocyanate; ethidium; fluorescein and derivatives such as 5-carboxyfluorescein (FAM), 5-(4,6-dichlorotriazin-2-yl)aminofluorescein (DTAF), 27-dimethoxy-4′5′-dichloro-6-carboxyfluorescein (JOE), fluorescein, fluorescein isothiocyanate (FITC), QFITC (XRITC); fluorescamine derivative (fluorescent upon reaction with amines); IR 144; IR 1446; Malachite Green isothiocyanate; 4-methylumbelliferone; ortho cresolphthalein; nitrotyrosine; pararosaniline; Phenol Red, B-phyco erythrin; o-phthaldialdehyde derivative (fluorescent upon reaction with amines); pyrene and derivatives such as pyrene, pyrene butyrate and Succinimidyl 1-pyrene butyrate; Reactive Red 4 (Cibacron® Brilliant Red 3B-A), rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl Rhodamine, tetramethyl rhodamine isothiocyanate (TRITC); riboflavin; rosolic acid and lathanide chelate derivatives, quantum dots, cyanines, pyrelium dyes, and squaraines. In some embodiments, the fluorophore comprises at least one alkene group (C═C) capable of forming an epoxide with a peroxy acid. In some embodiments, the fluorophore is a member of the Alexa Fluor™ family of fluorophores (e.g., Alexa 488, 532, 555, or 647). In one embodiment, the fluorophore is DyLight™ 750

An innovation of the current multiplex system involves the use of an optimized upright water dipping or water immersion objective. In embodiments, the system includes high numerical aperture (NA) (>1.0) objectives. In embodiments, a large field of view (FOV) (e.g., 16×0.8 NA, or 60×1.2NA) objective may be used. Water dipping objectives (e.g., 16×, 20×, with 0.8 NA objectives, 40×, 60× with 1.0-1.2NA) are typically used without a coverslip and with the tip of the objective dipped into water surrounding the sample, as opposed to water immersion objective which may be used with a coverslip having a drop of water on top, or in the present system without a coverslip and instead using a FPE viewing window. The objective is used to visualize a sample present in the imaging chamber that includes a refractive index matching optical window having the same refractive index as, for example, water.

The microscope further comprises a motorized stage that provided for highly repeatable movements and the integrated control of imaging, positioning and fluidics using software and hardware configured for that purpose. The use of such a motorized stage, and controllable imaging, positioning and fluidics provided the acquisition of tiled images of the sample.

### Multiplexing

Multiplexing refers to the use of more than one binding agent, which in turn means that more than one target molecule may be detected in the same sample, if present (although multiple binding agents may in principle bind to a single target molecule, e.g. where multiple antibodies bind to a large polypeptide, with each antibody binding to a different epitope). Multiplexing may therefore allow for the observation of multiple target molecules in parallel.

These binding agents are in general contacted with the sample e.g. at the same time and may bind to their respective targets, if present, in the same sample. An example of a standard multiplexed assay would be using a first primary antibody against one target molecule and a second primary antibody against a second target molecule. The sample and any bound primary antibody would then be contacted with the corresponding secondary antibodies which were labelled with fluorophores, a first fluorophore for the first secondary antibody and a second fluorophore for the second secondary antibody. By detecting any signal from each of the two fluorophores an image can be generated of the location within the sample of the two target molecules.

A multiplexing process therefore requires the ability to detect more than one fluorescent signal from the same sample. In standard fluorescent microscopy techniques this may be carried out by using more than one fluorophore, for example fluorophores that emit light of sufficiently different wavelengths to allow them to be differentiated. Suitable examples would be methods in which a fluorescein based molecule as a first fluorophore and a rhodamine based molecule is used as a second fluorophore.

### Sample

A biological sample disclosed herein can include any sample comprising a cell, a tissue, or a derivative of a cell or a tissue. In some embodiments, a biological sample herein has been treated to preserve analytes in the sample prior to analysis. In some embodiments, a biological sample herein includes a fixed cell or tissue sample comprising molecular crosslinks. The ability to use a fixed biological sample in an analytical method, such as in situ analysis of biological molecules (e.g., genomic DNA, RNA, cDNA, and/or proteins), can be enhanced in some cases if the cross-links established during fixation of the biological sample are reversed so that an assay can be carried out before sample degradation occurs.

In some embodiments, the biological sample is fixed and the fixation comprises contacting the sample with one or more agents that react with one another and/or with molecules in the biological sample. In some embodiments, the reaction creates molecular crosslinks between molecules of the one or more agents, between molecules in the biological sample, and/or between molecules of the one or more agents and molecules in the biological sample. In some embodiments, the one or more agents are crosslinking agents, and the molecular crosslinks are products of one or more reactions between a crosslinking agent and a molecule in the biological sample.

The sample will in general comprise one or more cells (e.g. a population of cells), or may comprise a cell lysate from a cell or a population of cells. The sample may contain prokaryotic or eukaryotic cells, e.g. animal, plant, yeast, bacterial or other cells. The cells are preferably animal cells. They may be of animal original and may in particular be of mammalian origin. They may be primary cells or may be cells that are a cell line, e.g. a mammalian cell line. The sample may contain cells that are from an animal model of a disease or a human or animal patient with a disease. The sample may be a biological sample, e.g. from an animal (preferably a mammal such as a human or an experimental animal e.g. a mouse or rat) and the sample may e.g. by blood, sputum, lymph, mucous, stool, urine and the like. The sample may be a tissue sample such as a tissue section. The sample may be an environmental sample such as a water sample, an air sample, a food sample, and the like.

### Fixation of the Sample

In some embodiments, the sample may be fixed prior to imaging. Fixation of samples for fluorescence microscopy is well known and is carried out in order to preserve the sample (e.g. to maintain cell morphology, in a state that is structurally close to its state when living). Fixation may also prevent the autolysis and necrosis of excised tissues, if used. It may also preserve antigenicity, and allow the components used in the methods to more readily access the internal portions of the cell.

Examples of suitable ways to carry out chemical fixation include, for example using crosslinking fixatives (e.g. to create covalent chemical bonds between protein in samples), such as those that are based on aldehyde, or oxidising crosslinking fixatives. Examples of aldehyde based fixatives include formaldehyde, glutaraldehyde, glyoxal, paraformaldehyde. Oxidising crosslinking fixatives for example osmium tetroxide, chromic acid, potassium dichromate, potassium permanganate. Precipitating fixatives may alternatively be used, for example acetone, ethanol, methanol. Alternative agents include Hepes-glutamic acid buffer-mediated organic solvent protection effect (HOPE) fixative, mercurial such as B-5 and Zenker's fixative, and picrates.

### Illumination

In some embodiments, the fluorescent emitter molecules are illuminated using an appropriate spectral emission, thereby causing the fluorescent emitter molecules to generate its predetermined fluorescent wavelength emission. This may also be referred to as “excitation” and an appropriate wavelength may be selected for this purpose based on the known properties of the fluorescent emitter molecule or combinations thereof that are being used. A single source may be used to excite multiple fluorescent emitter molecules, if used. Alternatively, a distinct source may be used for each fluorescent emitter molecule that is to be used, or a combination thereof may be used. Fluorescent emitter molecules have well characterised excitation or absorption spectra and selecting a suitable wavelength or combinations thereof for excitation of all of the fluorescent emitter molecules is well known in the art.

### Systems for Multiplex Imaging

Aspects and embodiments of the present disclosure provide systems for multiplex imaging. Exemplary systems of the disclosure are illustrated, with reference to the Figures, below.

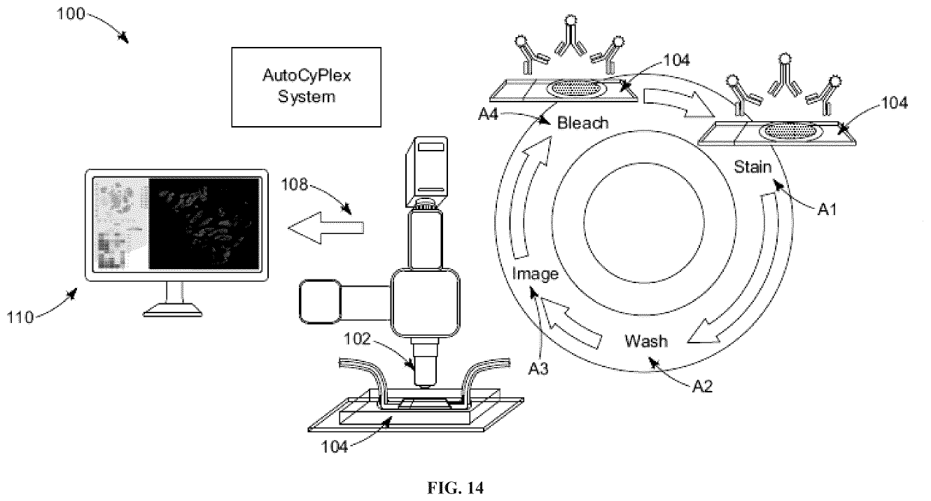

Referring to FIG. 14, a microfluidic system 100 is illustrated in the form of an “AutoCyPlex System” that is configured to automate cyclic multiplex protocols for formalin-fixed paraffin embedded (“FFPE”) tissue sections, including staining and imaging. The microfluidic system 100 increases the speed and extent of bleaching fluorophores for multi-round immunofluorescence staining approximately 10-fold compared, for example, to peroxide-based systems. Applications of the microfluidic system 100 include, for example, human and animal tissue staining across biomedical sciences.

The microfluidic system 100 includes a widefield upright microscope 102 for viewing a tissue slide 104 as it cycles through the automated cyclic multiplex protocol. The cycle of the of the automated cyclic multiplex protocol includes a staining step A1, a washing step A2, an imaging step A3, and a bleaching step A4. A camera 106 images the tissue slide 104 in step A3 and send image data 108 to a monitor display 110.

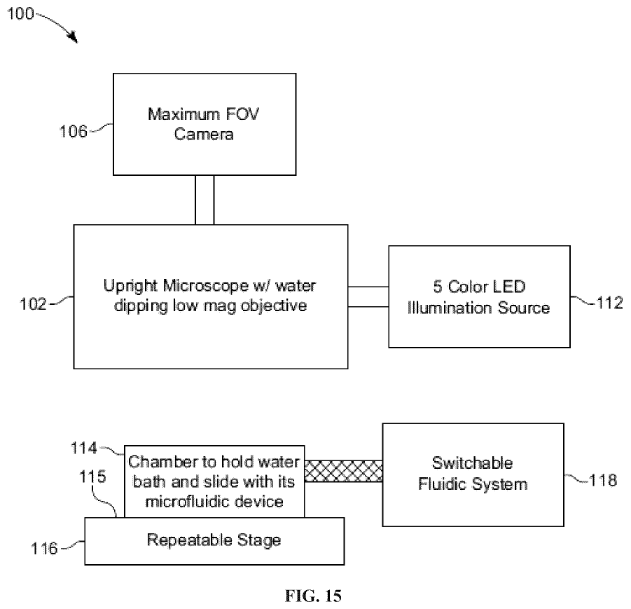

Referring to FIG. 15, the microfluidic system 100 uses, according to an example, a 5 color light-emitting diode (“LED”) illumination source 112. The illumination source 112 is communicatively coupled with the microscope 102 and the camera 106. Optionally, the camera 106 is a maximum field of view (“FOV”) camera, and the microscope 102 includes a water dipping objective.

The microfluidic system 100 further includes a fluid-immersion chamber 114 that holds, for example, a water bath and a slide with a microfluidic device. The fluid-immersion chamber 114 is positioned on a slide surface 115 of a repeatable stage 116. The fluid-immersion chamber 114 is in fluid communication with a switchable fluidic system 118. The repeatable stage 116 is, optionally, a high-resolution XY stage.

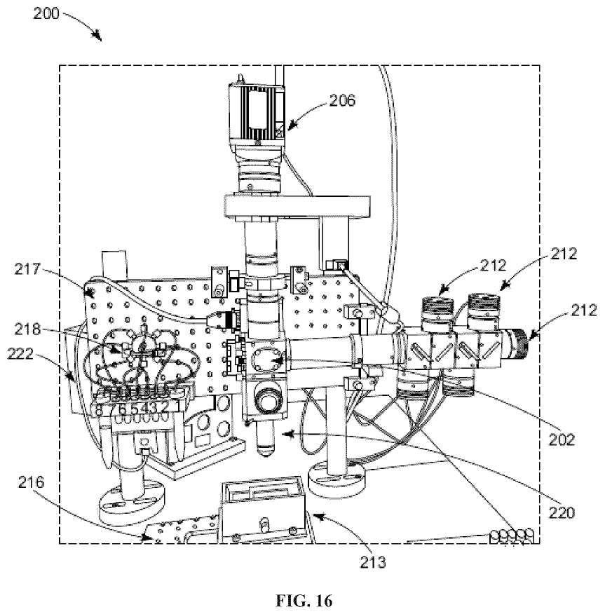

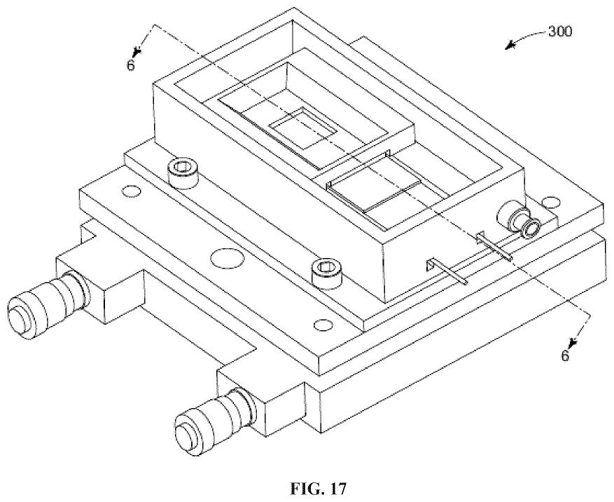

Referring to FIG. 16, a microfluidic system 200 includes widefield optics 202, which includes, for example, a widefield upright microscope and emission filters. The widefield optics 202 are communicatively coupled with a charge-coupled device (“CCD”) camera 206 for imaging tissue samples (as disclosed above in reference to FIG. 14). Excitation LEDs 212 provide illumination for the imaging of the tissue samples.

The microfluidic system 200 further includes a microfluidic chamber 213 that is positioned on a high-resolution XY stage 216, and an aluminum breadboard 217 on which the widefield optics 202 are mounted. The microfluidic system 200 further includes a fluidic system 218, which optionally includes a micro-peristaltic pump configured to control, via a multipinch valve, flow of fluorophores within the microfluidic chamber 213.

The widefield optics 202 include a high numerical aperture water dipping objective 220, which optionally includes objectives greater than 1.0 numerical aperture. The widefield optics 202 are communicatively coupled with a control board 222, which is configured to control an open-source microscopy control program via which the widefield upright microscope is operated.

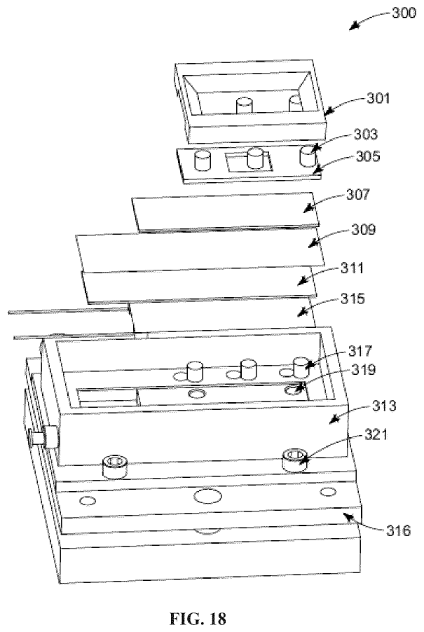

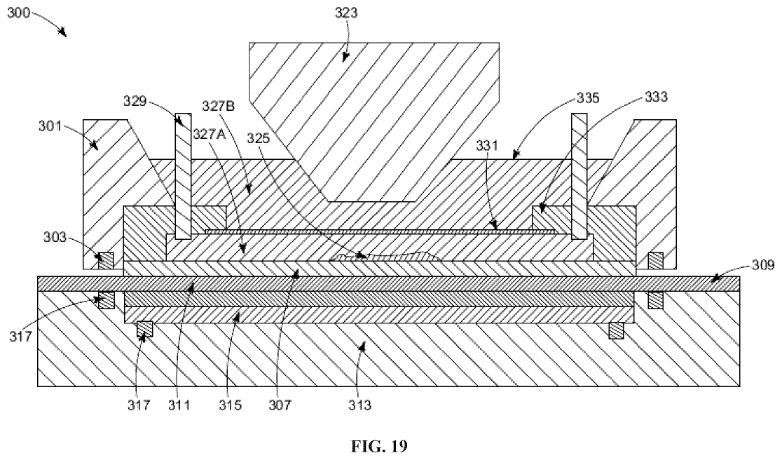

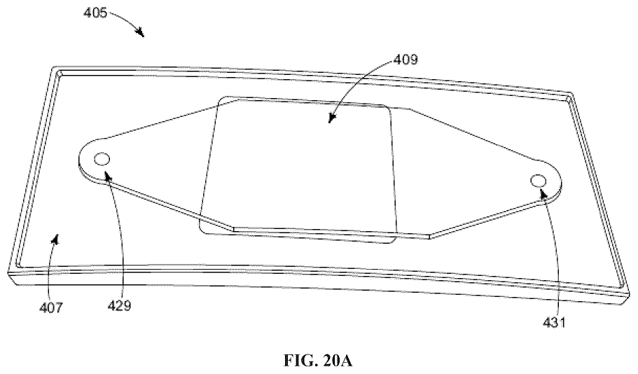

Referring generally to FIGS. 17-19, a microfluidic system 300 is further illustrated in accordance with other aspects of the present disclosure. Referring more specifically to FIGS. 18 and 19, the microfluidic system 300 includes a three-dimensional (“3D”) printed pressure plate 301 with pressure-plate embedded magnets 303 (e.g., neodymium magnets), which are positioned above an Invisi-Slip microfluidic device 305 (only shown in FIG. 18). A glass slide 307 is positioned below the microfluidic device 305 and above a maxi-black antireflective layer 309. A hard-setting potting compound (e.g., epoxy) 311 attaches the antireflective layer 309 to a positive temperature coefficient (“PTC”) heating element 315.

Pressure-plate chamber magnets 317 (e.g., neodymium magnets) and fluidic device stabilization magnets 319 are positioned in a 3D printed microfluidic chamber 313. The microfluidic chamber 313 is fastened to a stage 316 (only shown in FIG. 18) via a plurality of M6 mounting machine screws 321 (only shown in FIG. 18). Optionally, the stage 316 includes one or more of an XY stage and a tilt stage. One or more of the magnets 303, 317, 319 are optionally in the form of set screws that are adjustably embedded in locations directly adjacent (e.g., above or underneath) bonding walls of the microfluidic device 305. The set-screw form and shape is beneficial at least because it provides both adjustability and attachment for the magnets 303, 317, 319.

Referring more specifically to FIG. 19, the microfluidic system 300 includes a water dipping objective 323 for imaging a tissue sample 325 positioned on the slide glass 307. A fluidic medium 327A, such as phosphate buffered saline, fills the microfluidic chamber 333 such that the tissue sample 325 is completely immersed in the fluid 327A. Flow of the fluid 327A in and out of the microfluidic chamber 333 is facilitated via one or more tubes 329. The microfluidic system 300 further includes a fluorinated ethylene propylene (FEP) film 331 that is positioned above the tissue sample 325 and immersed within a fluid 327B. According to one example, the FEP film has the same refractive index as water, which when used in conjunction with a water immersion or water-dipping objective provides optically ideal imaging for an aqueous submerged tissue. One advantage of this type of FEP film, relative to other (existing) configurations, is that there are no refractive index mismatches (e.g., water/glass, glass/air). Thus, in contrast to these refractive index mismatches, which limit the resolution of imaging (limiting objective NA) or prevent imaging while the tissue is submerged, this disclosed example of FEP film is beneficial at least because it does not limit the resolution of imaging and does not prevent the imaging while the tissue is submerged.

The FEP film 331 separates the fluidic medium 327A from the fluid 327B. Thus, the fluidic medium 327A is in a completely enclosed fluidic area of the microfluidic chamber 333, while the fluid 327B is partially exposed to ambient surroundings near a top surface 335. The FEP film 331 extends internally between edges of a chamber body 333 that is made from a Polydimethylsiloxane (PDMS) material, which is embedded with iron oxide dust. Typically, PDMS is plasma treated to covalently bond to glass. However, the plasma treatment is not acceptable for multi-use purposes, because it makes slides functional only for single use. Effectively, plasma treatment makes slides useless after processing. Nevertheless, Van der Waals bonding between glass and PDMS provides an initial, albeit weak, bonding force that is a maximum of about 5 pounds per square inch (PSI).

Thus, the presently disclosed systems and methods add magnets (such as the magnets 303, 317 described above) to ensure leak protection in an effort-free and easy manner. The magnets are used alongside embedding iron oxide dust into the PDMS mixture, as described below. Optionally, the dust includes iron oxide nanoparticles or 320 grid dust (which is relatively inexpensive). According to one example, a good weight to weight ratio (w/w) is 1:3 PDMS to iron oxide.

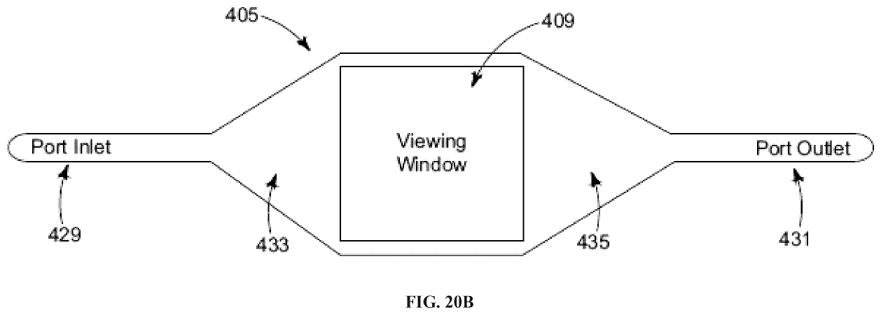

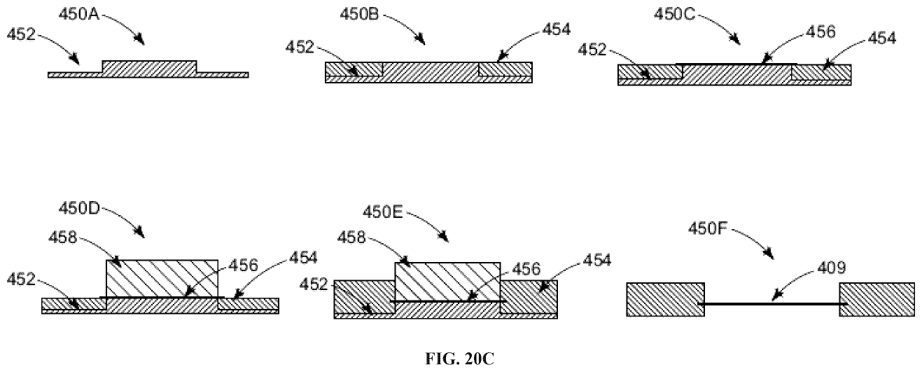

Referring to FIG. 20A, an Invisi-Slip microfluidic device 405 made has a generally rectangular chamber body 407 made from a PDMS material that is embedded with iron oxide dust. Within the chamber body 407, the microfluidic device 405 includes a viewing window 409 made from a FEP film. According to one example, the viewing window 409 has a rectangular shape that is approximately 15 millimeters (mm) long by 15 mm wide.

The FEP film of the viewing window 409 is sandwiched within the PDMS material of the chamber body 407. The iron oxide that is embedded in the PDMS is beneficial for magnetic alignment of the microfluidic device 405 when positioned within any of the microfluidic system (e.g., microfluidic systems 100, 200, 300) disclosed herein.

Optionally, the microfluidic device 405 further includes tubing ports 429, 431 for facilitating flow of a fluid through the microfluidic device 405. The tubing ports 429, 431, include an inlet port 429 and an outlet port 431 By way of example, the tubing ports 429, 431 receive within respective tubes 329 (shown in FIG. 19) for inlet and outlet of water.

Referring to FIG. 20B, according to an exemplary basic design configuration, the microfluidic device 405 includes the viewing window 409, the inlet port 429, the outlet port 431, and transition zones 433, 435 from the ports 429, 431 into the viewing window 409 area. According to one design criteria, an overall height from a slide glass (e.g., slide glass 307) is less than about 2 mm, which allows for 1.1 numerical aperture objectives to be used). According to another design criteria, a low-volume is facilitated over a tissue sample, such as about 70 micro-liters (μL) or less.

According to yet another design criteria, the viewing window 409 is optically “pure” (e.g., made from FEP film) to allow clear sight to tissue samples. According to yet another design criteria, the microfluidic device 405 provides reversible bonding.

According to yet another design criteria, the microfluidic device 405 is dimensioned to fit 75 mm×75 mm slides (e.g., slide glass 307) without touching an 18 mm frosted area. According to yet another design criteria, the ports 428, 43 are separated from the viewing window 409 spatially to prevent collision with the objective.

Referring to FIG. 20C, construction of the viewing window 409 is generally illustrated in six steps 450A-450F. Initially, it is noted that typically FEP material is difficult to adhere to any material. However, as disclosed in the present disclosure, this problem is being successfully overcome with the aspects disclosed herein. For example, using a FluoroEtch chemical material allows modification of the FEP material such that it can adhere to other materials. In step 450A, a silicon wafer mold 452 is made. In step 450B, initial PDMS material 454 is spin coated up to the height of the mold 452 and is partially cured. In step 450C, vacuum grease is applied to the mold 452 and, then, a FluoroEtch-treated FEP film 456 is placed onto the mold with a predetermined and slight overhang.

In step 450D, vacuum grease is applied to a bottom of a stereolithography (“SLA”) printed resin block 458 that has the same dimensions as the viewing window 409. The FEP film 456 is placed directly over the mold 452. In step 450E, additional PDMS 454 is poured onto the mold 452 until the desired height is reached, and then is fully cured. In step 450F, the SLA block, 458 which acts as a spacer, is removed from the mold 452. Then, the formed viewing window 409 is also removed from the mold 452 and placed in a Hexane bath to dissolve the vacuum grease and, thus, clean up the viewing window 409.

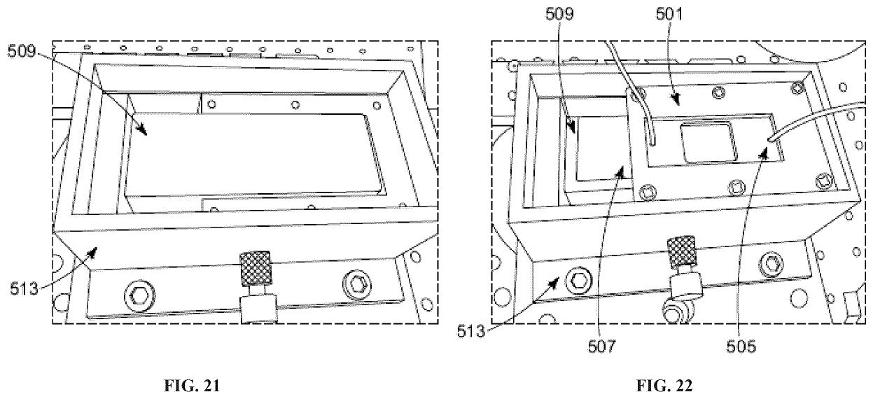

Referring to FIG. 21, a maxi-black antireflective layer 509 is shown positioned within a 3D printed microfluidic chamber 513. The antireflective layer 509 is mounted prior to adding additional components, as discussed below.

Referring to FIG. 22, a 3D printed pressure plate 501 is positioned and mounted within the microfluidic chamber 513. The 3D printed pressure plate 501 is mounted above a microfluidic device 505, which, in turn, is positioned above a slide glass 507. The slide glass 507 is positioned above the antireflective layer 509.

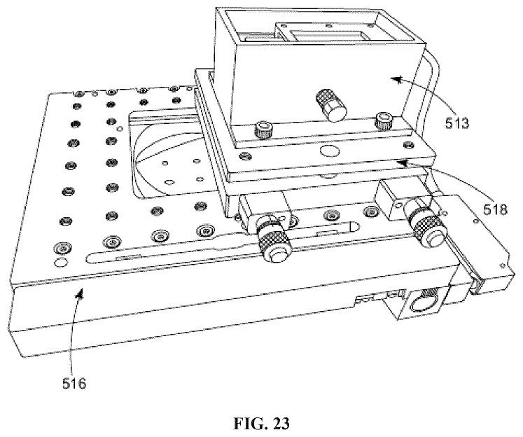

Referring to FIG. 23, the microfluidic chamber 513 is positioned on a plurality of stages 516, 518. Specifically, the microfluidic chamber 513 is mounted directly to a tilt stage 518, which is mounted directly to an XY stage 516. The tilt stage 519 facilitates, for example, a +/−3-degree tilt stage to level the slide glass 507 (shown in FIG. 22). The XY stage 516 facilitates, for example, large displacements of about 125 mm in both X and Y directions. More generally, the combination of the stages 516, 518 facilitates sub-pixel repeatability over a tissue section, allowing leveling and displacement of a tissue sample positioned on the slide glass 507.

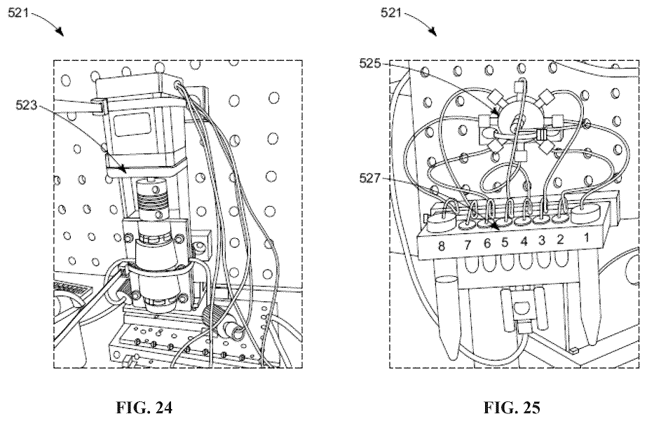

Referring generally to FIGS. 24 and 25, the microfluidic chamber 513 (shown in FIGS. 22 and 23) is in fluid communication with a fluidic system 521. Referring specifically to FIG. 24, the fluidic system 521 includes a micro-peristaltic pump 523 for fluid exchange in the microfluidic chamber 513. The micro-peristaltic pump 523 is optionally driven by a stepper motor. Referring specifically to FIG. 25, a multipinch valve 525 is shown in the form of an 8-channel pinch valve that is coupled to a multiplex mixer 527. The multipinch valve 525 and the multiplex mixer 527 facilitate addition of antibodies, a dye-inactivation buffer, and wash buffers.

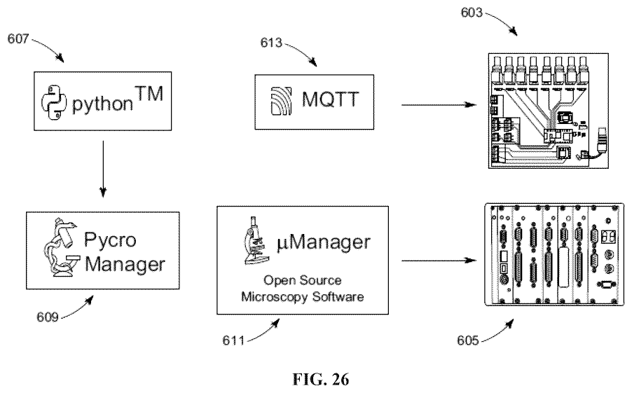

Referring to FIG. 26, the microfluidic systems disclosed herein are communicatively coupled and controlled via one or more of a fluidic control board 603 and a microscope control board 605. According to one example, the communication control is based on Python programming software 607. To control a microscope (e.g., microscope 102), the Python software 607 is used in a Pycro-Manager 609, which is an open-source software for customized and reproducible microscope control.

The Pycro-Manager 609 is further implemented in μManager 611, which is an open-source microscopy software that is dedicated to controlling microscopes, such as those manufactured by Leica, Nikon, Olympus, and Zeiss. The μManager 611 provides a simple and clean user interface, expediting execution of common microscope image-acquisition tasks, such as time-lapses, multi-channel imaging, z-stacks, and combinations thereof. The μManager 611 is configured to control the microscope control board 605, which is optionally an Applied Scientific Instrumentation (“ASI”) tiger microscope controller.

Separately, the fluidic control board 603 is controlled via a machine to machine network protocol MQTT 613. The MQTT protocol 613 is configured to control fluidic aspects of the fluidic control board 603.

In some embodiments, the system further includes and/or is configured for use with a composition for fluorescence inactivation (e.g., a composition including meta-chloroperoxybenzoic acid) of the present disclosure.

### Peroxy Acids

In embodiments, peroxy acids are used in systems, methods, or compositions for fluorescence inactivation and/or multiplex imaging.

A peroxy acid (also known as a peroxyacid or a peracid) is an acid which includes an acidic —OOH group. Peroxy acids have the general formula shown below:

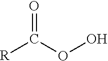

Without intending to be bound by theory, it is expected that the acidic —OOH group of the peroxy acid contacts an alkene group (C═C) of the fluorophore to form an epoxide, which contributes, at least in part, to inactivation of the fluorophore.

Exemplary peroxy acids include, without limitation: peracetic acid; m-CPBA; magnesium monoperphthalate; Payne's reagent; trifluoroperacetic acid; 2,4-dinitroperbenzoic acid; Caro's acid; and potassium caroate.

### Methods for Fluorescence Inactivation and Multiplex Imaging

The methods disclosed herein generally relate to inactivating fluorescence from a fluorophore. In some embodiments, the method includes contacting a composition comprising a compound that inactivates the fluorescence (e.g., a peroxy acid) with the fluorophore. In embodiments, the peroxy acid is: peracetic acid; m-CPBA; magnesium monoperphthalate; Payne's reagent; trifluoroperacetic acid; 2,4-dinitroperbenzoic acid; Caro's acid; and/or potassium caroate. The composition is basic and the composition inactivates fluorescence from the fluorophore. The inactivation occurs at a rate at least about two, three, four, five, six, seven, eight, nine, or ten times faster than inactivation with hydrogen peroxide under substantially the same or similar conditions. In embodiments, the composition inactivates the fluorophore at least about 2, 3, 4, 5, 6, 7, 8, 9, 10, 15, 20, 25, 30, 35, 40, 45, 50, 100, 150, 200, 250, 300, 350, 400, 450, 500, 550, 600, 650, 700, 750, 800, 850, 900, 950, or 1,000 times faster than inactivating with hydrogen peroxide or another inactivation agent under substantially similar conditions. Substantially similar conditions includes the use of the same buffer, the same fluorophore, the same temperature, and/or the same pH. In some embodiments, the pH used is about 9 or 10. The term “substantially similar conditions” does not require the same concentrations to be used. Instead, the normal usage concentration of each composition is used. For hydrogen peroxide, the normal usage concentration is 4.5% (v/v), or 1.47 molar (M), hydrogen peroxide. For m-CPBA, the normal usage concentration is 5 micromolar (μM). The normal usage of lithium borohydride (LiBH₄) is 10 millimolar (mM).

In some embodiments, the composition inactivates fluorescence without producing significant amounts of oxygen gas. In other embodiments, the composition inactivates fluorescence without producing detectable amounts of oxygen gas. In other embodiments, the composition inactivates fluorescence without producing amounts of oxygen gas that interfere or that substantially interfere with microscopic imaging. In still other embodiments, the composition inactivates fluorescence that produces amounts of oxygen that are 10, 25, 50, 75, or 100% less than the level of oxygen gas produced by inactivation with hydrogen peroxide under approximately the same or similar conditions. In some embodiments, the composition has a pH in a range of about 8 to about 12, in about 9 to about 11, in about 9.5 to about 10.5, and/or about 10. In some embodiments, a concentration of a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) in the composition is from about 1 micromolar to about 10 micromolar. In other embodiments, the concentration of a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) in the composition is greater than 10 micromolar.

In some embodiments, the fluorophore is a member of the Alexa Fluor™ family of fluorophores (e.g., Alexa 488, 532, 555, or 647). In some embodiments, the contacting is for from about 3 minutes to about 5 minutes, and/or from about 3 minutes to about 10 minutes. In some embodiments, the fluorophore is bound to a target molecule. In further embodiments, the fluorophore is bound to a target molecule via a target binding ligand. For example, the fluorophore is linked to a target binding ligand and the target binding ligand is bound to the target molecule. In some embodiments, the method includes binding the fluorophore to a target molecule.

In some embodiments of the method, the fluorophore is in a sample. In some embodiments, samples are biological samples. For example, the biological sample may be a biological cell, a tissue, and/or a formalin-fixed paraffin embedded (FFPE) tissue section. In some embodiments, the method further includes observing fluorescence from the fluorophore prior to contacting with the composition comprising a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate). In further embodiments, the method includes observing fluorescence from a second fluorophore after the inactivating step. In yet further embodiments, the method includes a step of inactivating fluorescence from the second fluorophore after observing fluorescence from the second fluorophore. The contacting with the composition including a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) may be in solution. It is contemplated that the solution is phosphate buffered saline (PBS), citrate, or any other suitable solution.

According to some embodiments, a method of detecting a target molecule in a sample includes binding a first fluorophore to a first target molecule in the sample and observing fluorescence from the first fluorophore. The method further includes inactivating the fluorescence from the first fluorophore by contacting a composition including a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) with the first fluorophore bound to the first target molecule. The composition is basic and the composition inactivates fluorescence from the first fluorophore at a rate at least five times faster than inactivating with hydrogen peroxide under substantially similar conditions. For example, with the same temperature, pressure, and pH, the composition including a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) would inactivate fluorescence from the first fluorophore at least five times faster than a solution of hydrogen peroxide would.

In some embodiments, the composition has a pH in a range of about 8 to about 12, of about 9 to about 11, of about 9.5 to about 10.5, and/or a pH of about 10. In some embodiments, a concentration of a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) in the composition is from about 0.5 to about 20 micromolar, from about 1 to about 10 micromolar, and/or above 1 micromolar. In some embodiments, the first fluorophore is alexa-488. In some embodiments, the contacting if for from about 2 minutes to about 15 minutes, from about 3 minutes to about 10 minutes, and/or from about 3 minutes to about 5 minutes.

In some embodiments, the first fluorophore is linked to a target binding ligand and is bound to the first target molecule via the target binding ligand. In some embodiments, the sample is a biological sample. For example, the biological sample may be a biological cell, a tissue, and/or a formalin-fixed paraffin embedded (FFPE) tissue section. In some embodiments, the observing occurs in a microfluidic device.

The method of detecting a target molecule in a sample, according to some embodiments, further includes binding a second fluorophore to a second target molecule in the sample. Optionally, the first and second target molecules are different. The method may further include observing fluorescence from the second fluorophore. In some embodiments, inactivating fluorescence from the second fluorophore includes contacting a composition including a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) with the second fluorophore bound to the second target molecule. The composition is basic and inactivates fluorescence from the fluorophore at a rate at least five times (5×) faster than inactivating with hydrogen peroxide under substantially similar conditions. For example, under substantially similar conditions, inactivation of alexa-488 requires contacting with the composition for less than 5 minutes but requires 30-60 minutes of contacting with hydrogen peroxide for the same extent of inactivation. Under substantially similar conditions, lithium borohydride may require contacting for more than 15 minutes. In some embodiments, the contacting with the composition is in solution. It is contemplated that the solution is phosphate buffered saline (PBS), citrate, or any other suitable solution. In embodiments, the composition inactivates the fluorophore at least about 2, 3, 4, 5, 6, 7, 8, 9, 10, 15, 20, 25, 30, 35, 40, 45, 50, 100, 150, 200, 250, 300, 350, 400, 450, 500, 550, 600, 650, 700, 750, 800, 850, 900, 950, or 1,000 times faster than inactivating with hydrogen peroxide or another inactivation agent under substantially similar conditions.

In some embodiments, the second fluorophore is linked to a target binding ligand and is bound to the second target molecule via the target binding ligand. The first and second fluorophores may be the same or may be different. The first and second target molecules may be the same or may be different.

According to some embodiments, a multiplex imaging method for detecting a plurality of target molecules in a sample includes the following steps: (a) binding a first fluorophore to a first target molecule and a second fluorophore to a second target molecule in the plurality of target molecules; (b) observing fluorescence from the first and second fluorophores; (c) inactivating the fluorescence from the first and second fluorophore by contacting a composition comprising a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) with the first and second fluorophore; (d) binding a third fluorophore to a third target molecule, and optionally a fourth fluorophore to a fourth target molecule, in the plurality of target molecules; and (e) observing fluorescence from the third and, if used, the fourth fluorophore. The first and second target molecules are different and the first and second fluorophore are different. The composition is basic and inactivates fluorescence from the first or second fluorophore at a rate at least five times (5×) faster than inactivating with hydrogen peroxide under substantially similar conditions. The third and fourth target molecules are different and the third and fourth fluorophore are different. In some embodiments, the method further includes: (f) inactivating the fluorescence from the third and, if used, the fourth fluorophore by contacting a composition including a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) with the third and fourth fluorophore; and (g) optionally repeating steps (d)-(f) one or more times. The composition is basic and inactivates fluorescence from the first or second fluorophore at a rate at least five times (5×) faster than inactivating with hydrogen peroxide under substantially similar conditions. The composition used in steps (c) and (f) may be the same or differ. For example, the compositions may differ in concentration and/or pH. In some embodiments, a pH of the composition is in a range from about 8 to about 12, from about 9 to about 11, from about 9.5 to about 10.5, and/or about 10. In some embodiments, a concentration of a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) in the composition is from about 1 micromolar to about 10 micromolar. The concentration of a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) in the composition may be greater than 10 micromolar.

In some embodiments, the fluorophore is alexa-488. In some embodiments, the contacting is for from about 3 minutes to about 5 minutes, and/or from about 3 minutes to about 10 minutes. In some embodiments, the fluorophore is bound to a target molecule. In some embodiments, the first fluorophore is linked to a target binding ligand and is bound to the first target molecule via the target binding ligand. Similarly, in some embodiments, the second fluorophore is linked to a target binding ligand and is bound to the second target molecule via the target binding ligand. In some embodiments, the third fluorophore is linked to a target binding ligand and is bound to the third target molecule via the target binding ligand, and in some embodiments, the fourth fluorophore is linked to a target binding ligand and is bound to the fourth target molecule via the target binding ligand.

In some embodiments of the method, the sample is a biological sample. For example, the biological sample may be a biological cell, a tissue, and/or a formalin-fixed paraffin embedded (FFPE) tissue section.

In some embodiments, the third target molecule and one of the first and second target molecules are the same. In some embodiments, the first, second, and third target molecules are different. In some embodiments, the fourth target molecule and one of the first and second target molecules are the same. In other embodiments, the first, second, and fourth target molecules are different. In some embodiments, the third fluorophore and one of the first and second fluorophores are the same. In other embodiments, the first, second, and third fluorophores are different. In some embodiments, the fourth fluorophore and one of the first and second target fluorophores are the same. In some embodiments, the first, second, and fourth fluorophores are different.

In some embodiments, the contacting with the solution including a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) occurs in solution. It is contemplated that the solution is phosphate buffered saline (PBS), citrate, or any other suitable solution. In some embodiments, at least one of steps (b) and (e) occurs in a microfluidic device. In some embodiments, (c) does not result in the production of oxygen gas. For example, step (c) may occur in a solution substantially free of bubbles throughout the occurrence of step (c).

### Compositions for Fluorescence Inactivation and Multiplex Imaging

In some embodiments, a composition for inactivating fluorescence from fluorophore includes a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) and a solvent. The composition is basic and inactivates fluorescence from a fluorophore at a rate at least five times (5×) faster than inactivating the same fluorophore with hydrogen peroxide under substantially similar conditions. In some embodiments, the composition further includes a buffering agent. Examples of buffering agents include acetate, histidine, phosphate, citrate, propionate, tricine, borate and tris(hydroxymethyl)aminomethane (tris), barbital, cacodylate, collidine, formate, maleic acid, succinate, and any other suitable buffering agent. In some embodiments, the buffering agent is a phosphate buffer. Example phosphate buffers may include at least one phosphate salt such as, but not limited to, sodium phosphate (e.g., sodium dihydrogen phosphate, disodium hydrogen phosphate, trisodium phosphate and sodium aluminum phosphate), potassium phosphate (e.g., potassium phosphate monobasic and potassium phosphate dibasic), rubidium phosphate, caesium phosphate, and ammonium phosphate, and/or at least one phosphoric acid such as, but not limited to, pyrophosphoric acid, triphosphoric acid, and orthophosphoric acid. In some embodiments, the composition is formulated for use in inactivating fluorescence from a fluorophore.

In some embodiments, the pH of the composition is in a range from about 8 to about 12, from about 9 to about 11, from about 9.5 to about 10.5, and/or about 10. In some embodiments, the concentration of a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) in the composition if from about 0.5 to 20 micromolar, from about 1 to 10 micromolar, and/or is greater than 1 micromolar. In some embodiments, the solvent is water, methanol, ethanol, acetone, dichloroethane, chloroform, dimethyl ether, diethyl ether, or any other suitable solvent. In some embodiments, the composition includes a peroxy acid (e.g., peracetic acid; m-CPBA; magnesium monoperphthalate; Payne's reagent; trifluoroperacetic acid; 2,4-dinitroperbenzoic acid; Caro's acid; and/or potassium caroate) and phosphate-buffered saline (PBS) and has a pH about 10. In some embodiments, the composition inactivates fluorescence from alexa-488 at rate at least 5× faster than inactivating with hydrogen peroxide under substantially similar conditions. In embodiments, the composition inactivates the fluorophore at least about 2, 3, 4, 5, 6, 7, 8, 9, 10, 15, 20, 25, 30, 35, 40, 45, 50, 100, 150, 200, 250, 300, 350, 400, 450, 500, 550, 600, 650, 700, 750, 800, 850, 900, 950, or 1,000 times faster than inactivating with hydrogen peroxide or another inactivation agent under substantially similar conditions.

### Kits

The disclosure further provides kits for fluorescence inactivation and/or multiplex imaging. The kits may include a composition of the present disclosure formulated for fluorescence inactivation, and instructions for using the composition for inactivating a fluorophore. The kit may further include a fluorophore, such as, for example, a member of the Alexa Fluor™ family of fluorophores (e.g., Alexa 488, 532, 555, or 647). Where the kit includes a fluorophore, the kit may also include instructions for use of the fluorophore in multiplex imaging, followed by inactivation of the fluorophore with the composition formulated for fluorescence inactivation in a sample. The instructions may be printed directly on the container (when present), or as a label applied to the container, or as a separate sheet, pamphlet, card, or folder supplied in or with the container. The instructions can be provided in digital form on a portable data storage medium (e.g., a compact disk or USB drive) or stored remotely on a server that can be accessed remotely.

In some embodiments, the kit comprises a sterile container which contains a composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding compositions and/or fluorophores of the present disclosure.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: Design of Improved Multiplex Imagining System

An end-to-end system for multiplex imaging, which addresses the drawbacks in the field, was developed based on a series of novel or optimized methods. The system and corresponding method of use was named Spatial Photo-inactivation Enhanced Cyclic Target REsolved multiPlexing (SPECTRE-Plex).

The system was broadly based around three modules. First, the system includes an optimized upright water dipping objective based imaging and fluidic system, including custom solutions for repeatable, iterative image capture. Second, the system includes a microfluidic imaging chamber system incorporating a refractive index matching optical window that enables imaging of tissues at a variety of resolutions including the use of high numerical aperture (>1.0) objectives in conjunction with repeated solution changes. Third, the system includes a novel, fast, non-gas producing dye inactivation method that reduces cycle times.

Application of this system for a 22-plex, 7 round panel staining in healthy and diseased human proximal intestinal tissue was demonstrated, including downstream analysis. In addition, additional applications of the method and system were developed, including direct tissue-based measurements of antibody binding kinetics for quantitative validation and assessment of antibodies and staining conditions.

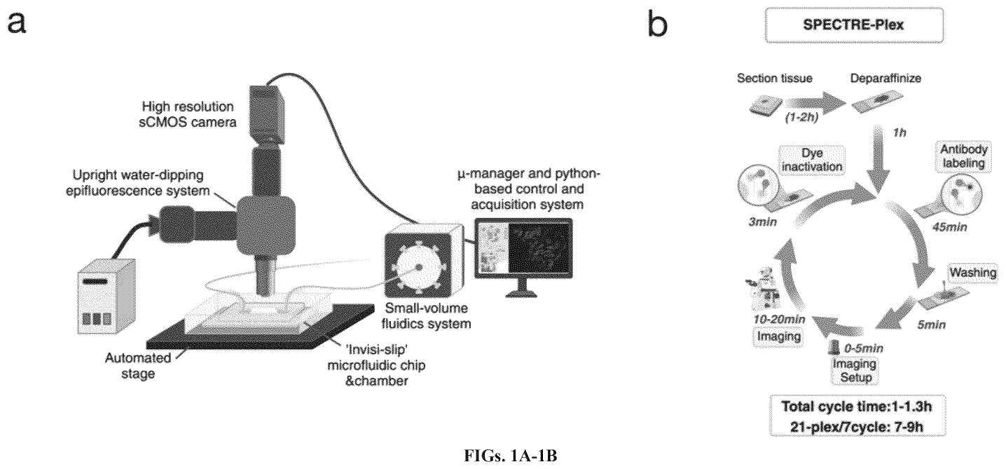

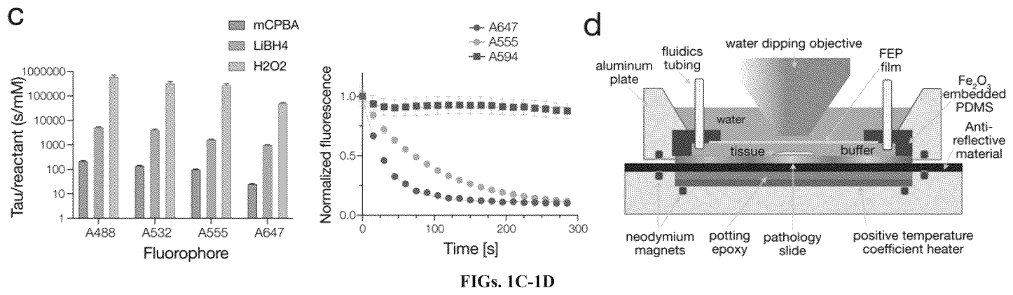

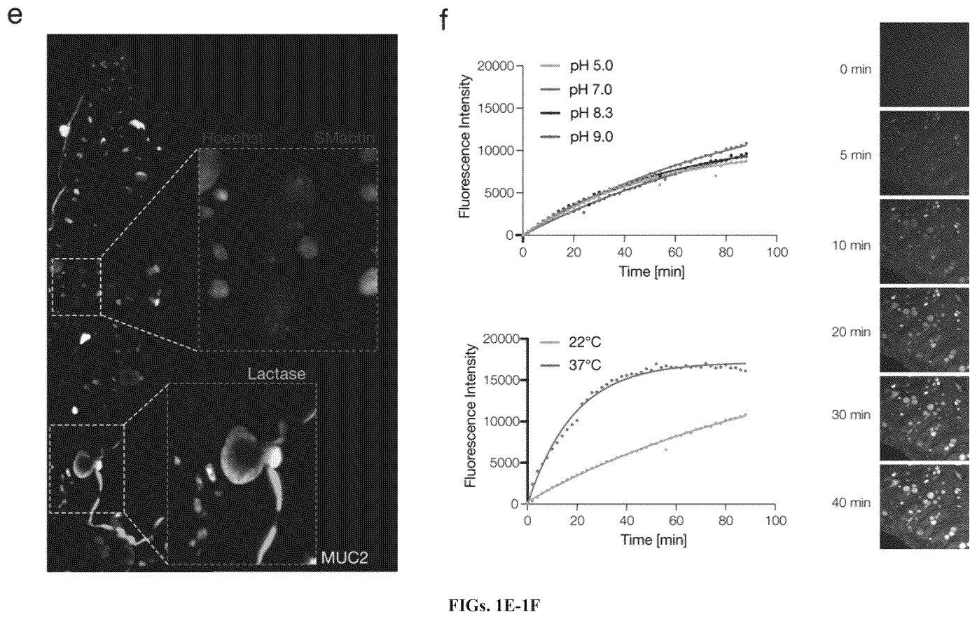

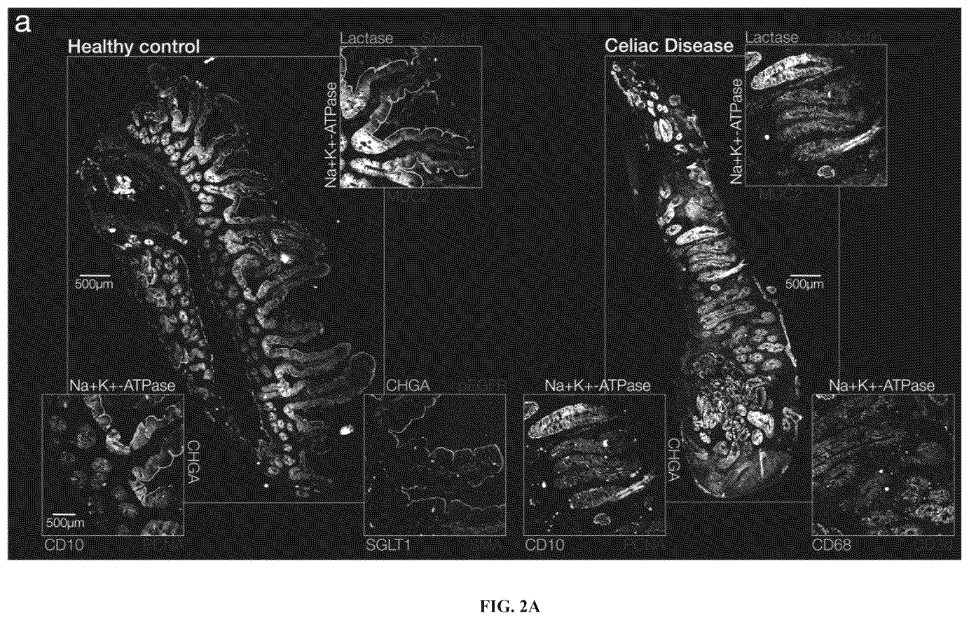

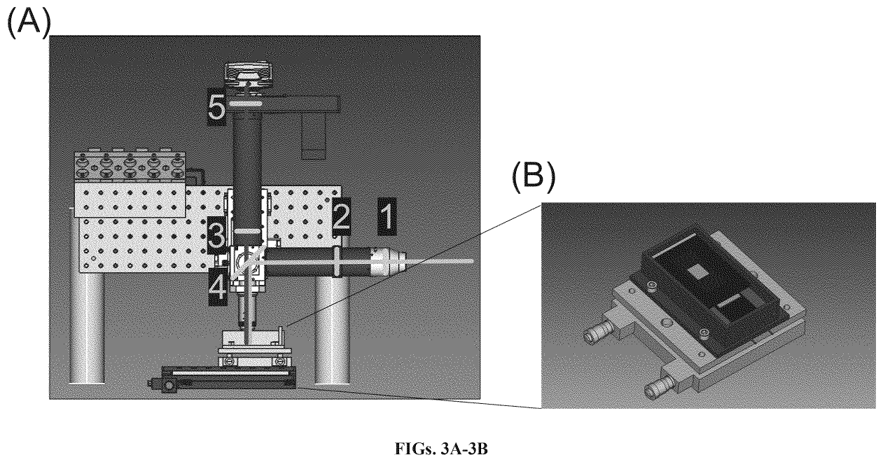

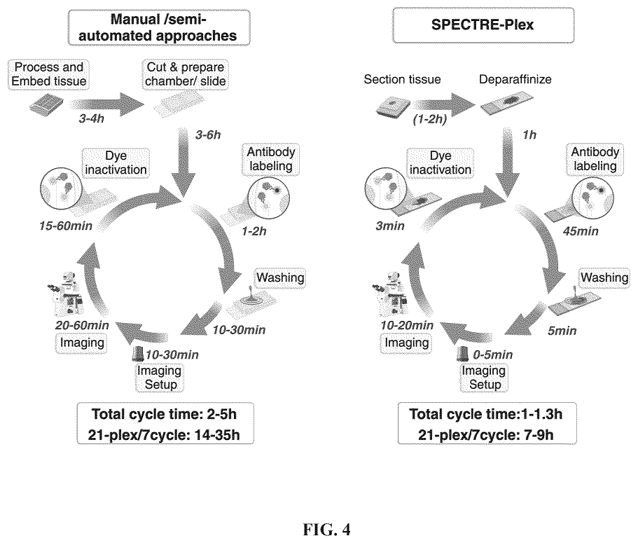

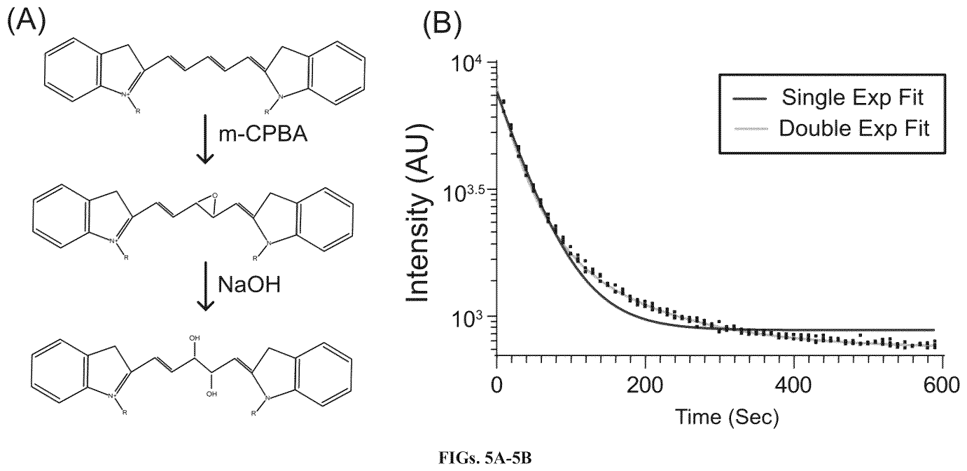

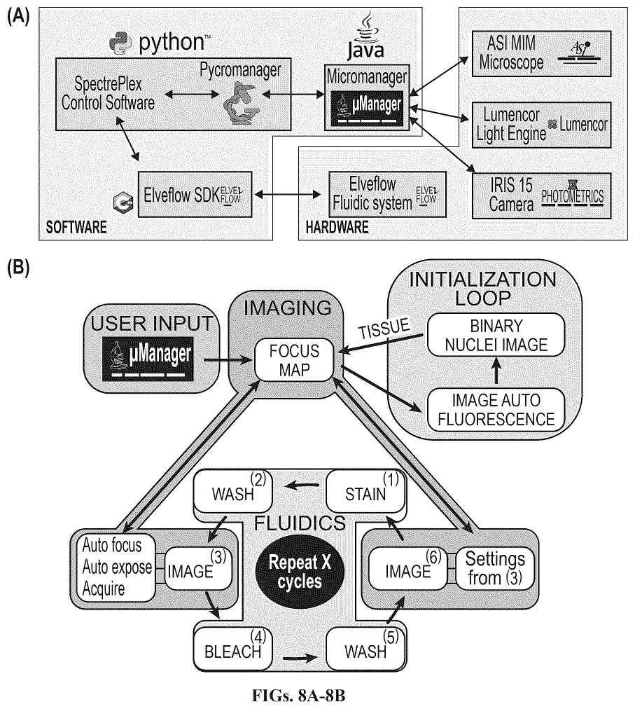

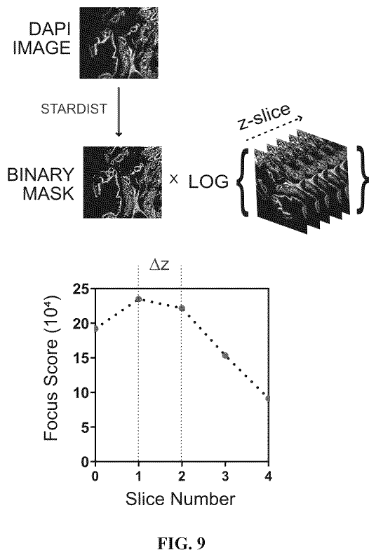

FIG. 1A and FIG. 3 outline the overall system set-up including custom optics with a water dipping objective, small volume fluidics system and python-based imaging and device control. The system was designed so that slide samples undergo staining, washing, imaging and dye inactivation in situ allowing efficient and rapid repetition of cycles (FIG. 1B). Overall, the method achieved a significant reduction in experimental time. As an example, a 7-cycle run could be completed in about 7 hours as compared to the approximately 14-36 hours that is required by most manual or semi-automated cyclical methods (FIG. 1B and FIG. 4). Tiled imaging in place was enabled by a highly repeatable motorized stage and integrated control of imaging, positioning and fluidics is achieved by custom python and μ-manager software (FIGS. 8-10).

### Example 2: Development of Novel Dye-Inactivation Method

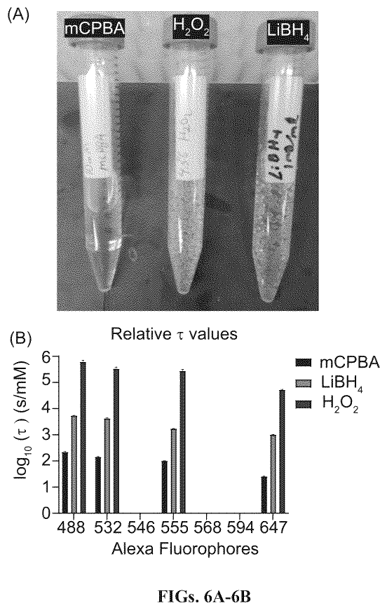

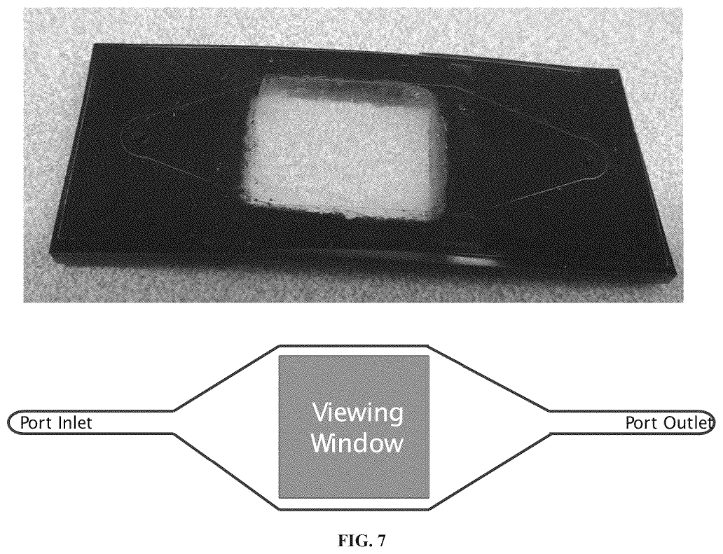

To further reduce cycle times the chemistry underlying inactivation of cyanine and rhodamine-based fluorophores was examined (FIG. 5). This led to the discovery of meta-chloroperoxybenzoic acid (m-CPBA) as a more efficient catalyst of the process. m-CBPA was tested against commonly used conjugated fluorophores (e.g. Alexa 488, 555 and 647) in parallel to the previously used dye-inactivation agents, lithium borohydride (LiBH₄) and hydrogen peroxide (H₂O₂) and it was found that dye inactivation by m-CPBA was orders of magnitude faster (FIG. 1C, FIG. 6). In addition, m-CPBA did not generate oxygen in solution, overcoming a major limitation of LiBH₄ and H₂O₂ for use in imaging and for the application of microfluidics. It was found that m-CPBA did not cause appreciable deterioration of tissue or image quality (tested up to 10 cycles) and the lack of bubble formation allowed easy implementation of small volume fluidic flow control.

### Example 3: Development of PDMS-Based Microfluidic Device

Imaging resolution and magnification is largely limited for current automated cyclical fluorophore staining methods because of the use of air objectives (usually NA≤0.8) and/or the use of glass chamber windows which introduce refractive index mismatching. To solve this issue, a PDMS-based microfluidic device (‘invisi-slip’) was developed which included an embedded fluorinated ethylene propylene (FEP) window enabling aberration free imaging as the FEP polymer had the same refractive index as water and therefore could be coupled to water-based objectives. This device was incorporated into an integrated imaging chamber system which included ports and tubing for fluids and temperature control (FIG. 1D). For whole tissue imaging and to reduce imaging area/tile size a large field of view (FOV) 16×0.8 numerical aperture water dipping objective was used. To illustrate the wide range of objectives that could be used, SPECTRE-plex runs were also carried out using a 60×1.2NA water dipping objective which allowed imaging of target proteins at sub-cellular resolution (FIG. 1E).

### Example 4: Testing of Antibody Kinetics Using SPECTRE-plex

The in-situ nature of the SPECTRE-plex design allowed for contemporaneous imaging and conjugated antibody labelling on tissues. This enabled experiments to investigate the kinetics of antibody-target protein association directly on tissues. To illustrate this, the kinetics of binding of a specific antibody (anti-Muc2 IgG) and the effect of altering solution pH or temperature was tested. As shown in FIG. 1f, at 22° C., anti-Muc2 exhibited a one phase association with a mean half-time of 50 mins, which was not altered by solution pH but was significantly reduced (13 mins) by increasing solution temperature to 37° C. This capability of the SPECTRE-plex system may be therefore be used for a variety of applications including, without limitation, tissue specific validation of antibodies and rapid assessment of therapeutic antibody or small molecule binding.

### Example 5: Multiplex Imaging of Celiac Disease Using SPECTRE-Plex

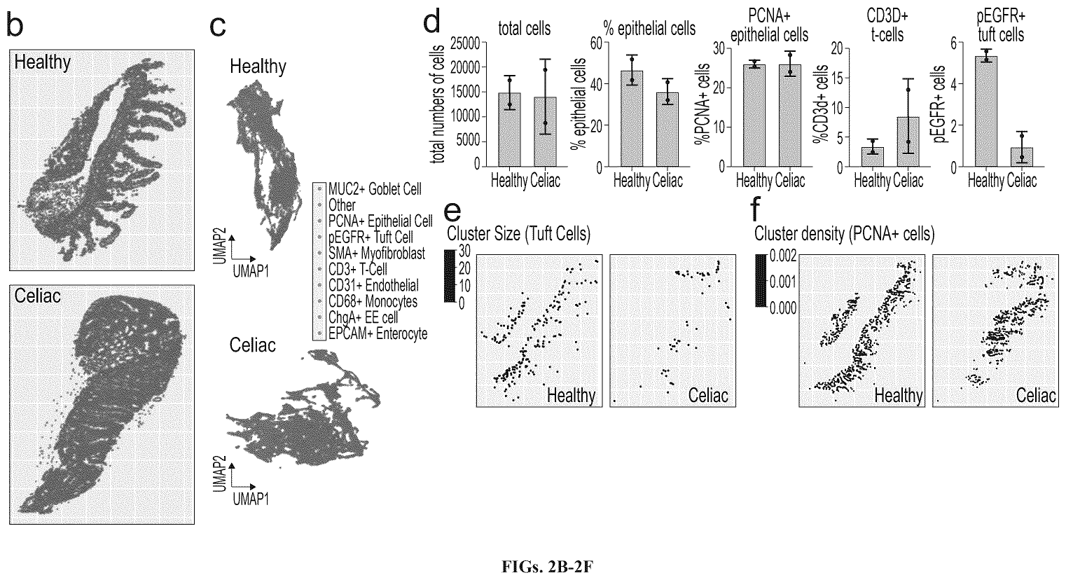

To demonstrate the use of the system a 22-plex image data set was generated using duodenal tissues obtained from children with active celiac disease and compared to age-matched healthy control duodenum. FIG. 2A shows representative registered and stitched whole tissue SPECTRE-plex images with examples of specific antigens including markers of specific cell types such as epithelial cells (Na⁺K⁺-ATPase), T-cells (CD3d), enteroendocrine cells (ChromograninA), and myofibroblasts (smooth muscle actin) among others (Table 1). A custom image analysis pipeline involving adaptive segmentation and cell coordinate mapping (FIG. 2B, FIG. 12) allowed for several downstream analyses. Cell compositional analysis (FIGS. 2B-2D) showed interesting differences between healthy and celiac disease tissue including a significant decrease in intestinal tuft cells, a lineage of epithelial cells involved in luminal nutrient and pathogen sensing (Billipp et al., Immunity S1074761324001444 (2024)). Celiac tissue also showed well described immune cell expansion and infiltration and a generally more heterogenous structure (FIG. 2C). To understand the spatial relationships among cell types, a neighborhood cell cluster analysis was performed for pEGFR+ tuft cells which indicated the presence of larger spatial clusters of tuft cells in healthy tissues (FIG. 2E). Cluster density analysis for PCNA+ cycling epithelial cells (FIG. 2F) showed focal areas of high density in celiac disease suggesting active regenerative zones.

Future improvements to SPECTRE-plex include the addition of a multi-slide configuration to enable high throughput experiments and screening, and addition of confocal optics to enable three-dimensional sample imaging. Although SPECTRE-Plex was primarily designed for dye-inactivation multiplex methods, the imaging and microfluidic modules can be readily applied to other tissue-based imaging including spatial transcriptomics protocols and other use cases such as quantitative antibody validation studies.

### METHODS OF THE EXAMPLES

The following methods were employed in the above examples.

### Imaging Chamber Design

The optical train for the SPECTRE-Plex is a custom design using the modular infinity microscope platform from Applied Scientific Imaging (ASI). This is an upright 4F widefield configuration with a Kohler illuminated, liquid light guide fed excitation path and an additional extra-long Nikon tube (265 mm vs 200 mm) to increase magnification to eliminate vignetting. The excitation source is a Lumencor light engine (Spectra III) containing integrated excitation filters, with a Photometrics IRIS 15 camera and Nikon CF175 series 16×0.8NA water dipping objective. The fluidic system comprises of an Elveflow OB1 mk4 air pressure regulator with inline flow meter, Elveflow multiplexing valve (12×1) and a custom 3D printed device to hold and organize the individual Eppendorf reservoir adapters. The stage is a combination of an ASI XY OE 1250 stage and a TechSpec tilt stage. The XY stage has a high travel range and is highly repeatable for positioning. The tilt stage allows for fine tuning of the chamber tilt to be parallel with the camera imaging plane.

The main body of imaging chamber is 3D printed out of PLA and has two sets of glued in neodymium magnets. One set of magnets is to interface with the pressure plate, while the other set is to aid in the alignment of the Invisi-Slip microfluidic device. A custom positive temperature coefficient (PTC) heater from Thermo-heaters LLC is adhered to main chamber body and a hard-set potting epoxy is poured over the heater and the magnets and pressed flat until it set. This encases the magnets and heater in epoxy, isolates them from the water in the chamber and enables good thermal conductivity. This also provides a very flat surface for the slide to rest on and seals the pores in PLA of the chamber body to make it watertight. The next layer, a polymer film called Maxi Black (Acktar) abrogates reflection in the system by absorbing a wide band of frequencies and preventing spectral scattering. A PLA 3D printed alignment jig allows reproducible placement of both the slide's positions in the chamber and placement of the pressure plate over the slide. Finally, the pressure plate was milled out of aluminum (Protolabs) and anodized to prevent any oxidation and contains sockets for neodymium magnets. The magnets were encased in a poured UV curable resin. FIG. 3 shows the imaging chamber design.

### Standard SPECTRE-Plex Protocol

### Reagents:

Histoclear (ThermoFisher Scientific, cat no. 50-329-48)
Ethanol (Decon Laboratories, cat no. 2716)
PBS (Corning, cat no. 21-040-CV)
Hoechst (ThermoFisher Scientific, cat no. H3570)
LiBH4 (Strem 9300397)
H2O2 (Sigma 216763)
m-CPBA powder (Sigma 273031)
bovine serum albumin (Sigma-Aldrich, cat no. A6003-25G)
Triton-X-100 (Sigma-Aldrich, cat no. X100-100ML)
Citrate stock solution (Abcam, cat no. ab93678)
DAKO Diluent (Agilent Dako, cat no. S080983-2)
Water

### Buffer Preparation

Bleach base solution: 24 mM NaOH was prepared by diluting 10M NaOH stock in PBS.
Blocking Buffer: 0.1% Triton-X-100 and 5% bovine serum was added to PBS.
Antigen Retrieval Buffer: The Citrate stock solution was diluted 100-fold in PBS. m-CPBA Bleach Solution: m-CPBA stock was prepared by dissolving m-CPBA powder in 100% ethanol at 1M concentration or 172 mg/mL. This stock was used for up to 30 days. For the working bleach solution, the stock was diluted 1:100 in bleach base solution.
LiBH₄ Bleach Solution: Dissolve the appropriate amount of LiBH₄ in distilled water for desired concentration (10 mM for bleach kinetics and 50 mM for bubble generation demonstration). The solutions were made from powder opened within the previous 30 days and used immediately after preparation.
H₂O₂ Bleach Solution: 30% H₂O₂ (Sigma 216763) was added to bleach base solution to make 4.5% H₂O₂.
Antibody Dilutions: Prepare 500 uL antibody dilutions from DAKO Diluent containing 1:25k dilution of Hoechst and desired antibodies for a given staining round.
Phosphate-Citrate Buffer for pH experiments: 100 mM concentration was used for all pH experiments. Buffers were made by adding appropriate amount of Sodium Phosphate Dibasic Dihydrate and citric acid. Sodium Hydroxide and Hydrochloric acid were used for fine pH adjustment. Ionic strength was calculated to be within 5% for each chosen pH value.

### Slide Preparation:

1. For deparaffinization, place slides in a 60° C. bead bath for 15 min.
2. Incubate with 100% Histoclear for 5 min at RT. Repeat this step twice with fresh Histoclear each time for 5 min and then 10 min at RT.
3. For rehydration, incubate slides in decreasing concentrations of Ethanol.
   a) First, incubate in 100% Ethanol for 5 min at RT. Repeat this step with fresh Ethanol for 5 min at RT.
   b) Incubate in 95% Ethanol for 5 min at RT. Repeat this step with fresh Ethanol for 5 min at RT.
   c) Incubate in 75% Ethanol for 5 min at RT.
   d) Incubate in 50% Ethanol for 5 min at RT.
   e) Incubate in tap water for 5 min at RT.
4. Move slides into a plastic Coplin jar and pour Antigen Retrieval Buffer in until tissue on slides is completely covered.
5. Submerge the Coplin jar into a boiling water bath for 20 min.
   The set-up is a 1 L beaker of boiling water on the heating plate with a stripette on the top to which the holder is attached with a binder clip.
6. Remove the Coplin jar from water bath and let cool at RT for 20-40 mins.
7. Wash with 1×PBS for 5 min. Repeat this step with fresh 1×PBS.
8. Pour Blocking Buffer into the Coplin jar and let sit at RT for 1 hour.
9. Store in 1×PBS at 4° C. until ready to stain.

### Multiplex Set up:

10. When ready to start SPECTRE-plex run, stain slide with 1:5k Hoechst dilution for 5 minutes. Give slides a quick rinse with 1×PBS.
11. Prepare the antibody dilutions (described above).
12. Mount a slide into the base of the chamber and place the fluidic device on top. Carefully lower the top plate making sure the fluidic device stays in desired placement. Insert the tubing onto either side of the fluidic device.
13. Check if a seal has been created between the slide and fluidic device by running PBS through tubing.
   a) Note: If PBS does not flow, then the seal has not been created. Remove the tubing and reseat the top plate if this occurs and test again.
14. Move the chamber so it is positioned underneath the objective. Open the Micro-manager software and lower the objective.
15. Locate the sample using the DAPI channel. Check if the sample is level.
16. Use the MicroMagellen plugin to define a surface.
17. Give the project folder a name and start the program.

### Comparison of Different Bleach Solutions

The following fluorophores were evaluated for their propensity to photobleach under different bleach solution:
1. Alexa-488 (Invitrogen prod #A28175, 1 mg/mL)
2. Alexa-532 (Life Technology A11002, 2 mg/mL)
3. Alexa-546 (Phallodin)
4. Alexa-555 (Invitrogen A21428, 2 mg/mL)
5. Alexa-568 (Life Technology A11031, 2 mg/mL)
6. Alexa-594 (Chromogranin)
7. Alexa-647 (Life Technologies A21246, 2 mg/mL)

Bleach solutions were prepared as described. All experiments were conducted as triplicates in a 96 well format using in a Tecan Spark multi-well plate imaging system in inverted fluorescence mode with the monochromator positions chosen to have 25 nm bandwidths centered on excited and emission peaks for each fluorophore. Each fluorophore was diluted at 1:1000 in PBS and with the appropriate concentration of each of the different bleach solution with the bleach solution without any fluorophores as their respective controls. The average fluorescence intensity normalized to the control was measured every 15 seconds for 10 minutes. The resulting kinetic data was fit in Graphpad Prism using a double exponential decay model.

### Antibody Staining Kinetics

The kinetics experiment was conducted on the SPECTRE-Plex system and modified from the standard SPECTRE-Plex imaging protocol. A slide stained with DAPI was loaded onto the microscope's stage and washed with PBS. For these experiments, a fixed exposure time and illumination intensity (50% power and 50 ms exposure time for both Alexa-488 and Alexa-647) was used. An initial image was taken as the background autofluorescence image and then the fluidic system was used to dispense the stain solution. Next, every 2 minutes, the system would execute the stardist recursive autofocus algorithm (as described in the autofocus section) and each channel imaged. A final image was taken 90 minutes after a PBS wash. Temperature and pH were modulated for these kinetic experiments. For the pH experiments, Phosphate-citrate buffer was used due to its large buffering range. Temperature was modulated using an embedded heater in the imaging chamber. For these experiments, the heater was then turned on for 1.5 hours in order to hit a stabile temperature which was measured by an infrared thermometer prior to antibody staining.

Images were processed by subtracting the background, zero-time frame and the average pixel intensity of the tissue region was calculated for each frame. A binary tissue mask was generated by thresholding the final background-subtracted image via Otsu's method. An exception was made for the pH 5.0 for MUC2 data as the signal was too low for Otsu's method to threshold accurately. In this case, the mask was set by manually tracing out 3 small regions of goblet cells in the image. The temporal change in average pixel intensity data was fit to a single exponential function in Graphpad Prism.

### Assessment of Fluidic Bubble Generation

H₂O₂, LiBH₄ and m-CPBA were mixed at normal working concentrations (4.5%, 50 mM, 10 mM respectively) in 15 mL tubes, vortexed and left to sit for 20 minutes for observing the propensity of each solution to form bubbles.

Comparison of mCPBA Over LiBH₄ and H₂O₂ for Dye Inactivation and Oxygen Production

mCPBA has three advantages over LiBH₄ and H₂O₂ for dye-inactivation and multiplex immunofluorescence applications. Firstly, mCPBA unlike both LiBH₄ and H₂O₂ does not produce air bubbles for up to 24 hours on incubation with target fluorophores (FIG. 6A). Absence of air bubbles is important for small volume microfluidic applications as these can rapidly accumulate in any closed system either in tubing or in the imaging chamber. This leads to sub-optimal and variable image quality as well as variability in fluid flow control for solution exchange. Secondly, mCPBA has much higher chemical and solution stability than LiBH₄ or H₂O₂, which are known to be relatively labile. Working concentrations of mCPBA continue to have sufficient efficacy for up to 36 hours and a 1M stock solution of mCPBA in ethanol is stable for 30 days. Thirdly, dye inactivation by mCPBA is orders of magnitude faster compared to LiBH₄ and H₂O₂. For all the antibodies that were tested, mCPBA was at least 10 times faster in bleaching than LiBH₄ and H₂O₂ at the recommended working concentrations (FIG. 6B).

### Invisi-Slip Microfluidic Device

Cover-slipping is an important engineering hurdle to overcome for automating multiplexing microscopy assays as the act of removing a coverslip, while trivial by hand, would require a robotic arm system to accomplish in an automated system. Most commercial automated platforms integrate these coverslips into the device that seals around the sample of choice, but this is often not readily adaptable for use in other systems. In addition, the use of coverslips and the consequent refractive index mismatching that occurs between fluid, glass and air results in inherent limitations to the numerical aperture and magnification of the imaging objective and therefore limitations to resolution. The Invisi-slip system circumvents these issues by using a Fluorinated ethylene propylene (FEP) polymer viewing window embedded in a PDMS device as a backbone. The PDMS backbone can be manufactured in any microfluidic device manufacturing facility that uses silicon wafer molds. The device is not plasma treated and is completely reusable. A magnetic pressure plate is used to seal the device to a slide. The 6 neodymium magnets in the pressure plate and 6 in the chamber below them with a 2 mm gap between them, easily gives enough sealing force to have fluid move 1000 uL/min within the device. The FEP viewing window can be made to be very large to accommodate large tissue sections or tissue microarrays. In the present case, the window has dimensions of 18 mm×21 mm. The internal volume of the device is approximately 120 uL which allows the system to use reagent quantities that align with microliter volume fluidics systems. Overall, this system is a reusable, flexible solution that allows for aberration free imaging as the FEP polymer has the same refractive index as water and therefore can be coupled to water immersion or dipping objectives. Furthermore, the small distance from the FEP film window to tissue sample, allows for the use of high numerical aperture (>1.0), or high magnification objectives. The design of the invisi-slip system is shown in FIG. 7.

TABLE 1
List of antibodies used

| Cycle | Marker | Clone | Conjugate | Isotype | Vendor | Cat No. | Dilution |
|---|---|---|---|---|---|---|---|
| 1 | Hoechst | — | — | — | ThermoFisher | H3570 | 1:25000 |
| | Chromogranin A | CGA/413 | Alexa Fluor | IgG2b | Novus | NBP2- | 1:500 |
| | | | 488 | Kappa | | 47850AF488 | |
| | Sodium | EP1845Y | Alexa Fluor | IgG | Abcam | AB274883 | 1:250 |
| | Potassium | | 555 | | | | |
| | ATPase | | | | | | |
| | pEGFR | EP774Y | Alexa Fluor | IgG | Abcam | AB205828 | 1:100 |
| | | | 647 | | | | |
| 2 | Hoechst | — | — | — | ThermoFisher | H3570 | 1:25000 |
| | SGLT | SLC5A1 | Alexa Fluor | IgG | Novus | 41858 | 1:50 |
| | | | 488 | | | | |
| | Pan CytoKeratin | AE1/AE3 | eFluor 570 | IgG1 | ThermoFisher | 2676893 | 1:100 |
| | FoxP3 | 206D | Alexa Fluor | IgG1 | BioLegend | B408885 | 1:50 |
| | | | 647 | Kappa | | | |
| 3 | Hoechst | — | — | | ThermoFisher | H3570 | 1:25000 |
| | Smooth Muscle | EPR5368 | Alexa Fluor | IgG | Abcam | AB202295 | 1:250 |
| | Actin | | 488 | | | | |
| | CD45 | — | Alexa Fluor | IgG | CST | 19581S | 1:50 |
| | | | 555 | | | | |
| | CD14 | EPR21847 | Alexa Fluor | IgG | Abcam | ab302829 | 1:20 |
| | | | 647 | | | | |
| 4 | Hoechst | — | — | | ThermoFisher | H3570 | 1:25000 |
| | CD4 | — | Alexa Fluor | IgG | R&D Systems | FAB8165G | 1:50 |
| | | | 488 | | | | |
| | CD68 | — | Alexa Fluor | IgG | CST | 23308S | 1:50 |
| | | | 555 | | | | |
| | MUC2 | EPR21847 | Alexa Fluor | IgG | Abcam | ab311136 | 1:1000 |
| | | | 647 | | | | |
| 5 | Hoechst | — | — | | ThermoFisher | H3570 | 1:25000 |
| | Lactase | 4E10 10.2 | Alexa Fluor | IgG1 | Novus | NBP2- | 1:250 |
| | | | 488 | | | 50513AF488 | |
| | CD3D | EP4426 | Alexa Fluor | IgG | Abcam | ab208514- | 1:100 |
| | | | 555 | | | 1001 | |
| | gamma actin | 1-17 | Alexa Fluor | IgG21 | SCB | sc-65638 | 1:100 |
| | | | 647 | Kappa | | AF647 | |
| 6 | Hoechst | — | — | | ThermoFisher | H3570 | 1:25000 |
| | CD10 | F-4 | Alexa Fluor | IgG1 | SCB | sc-46656 | 1:50 |
| | | | 488 | Kappa | | AF488 | |
| | PDGF Receptor | | Alexa Fluor | | CST | 79304BC | 1:100 |
| | Beta | | 555 | | | | |
| | CD31 | EPR3094 | Alexa Fluor | IgG | Abcam | ab218582 | 1:20 |
| | | | 647 | | | | |
| 7 | Hoechst | — | — | | ThermoFisher | H3570 | 1:25000 |
| | PCNA | PC10 | Alexa Fluor | IgG2a | SCB | sc-56 AF488 | 1:20 |
| | | | 488 | Kappa | | | |
| | EpCAM | EPR677(2) | Alexa Fluor | IgG | Abcam | ab275122 | 1:250 |
| | | | 555 | | | | |
| | Lysozyme | BGN/0696/5B1 | Alexa Fluor | IgG2a | Novus | NB100- | 1:100 |
| | | | 647 | | | 63062AF647 | |

### Acquisition Outline

An initial acquisition is performed to capture autofluorescence with default exposure times. The DAPI images are segmented as a binary image using Stardist and run through a size exclusion filter. The focus map is updated with this information for each tile to image only the tiles where tissue exists. Next, in the cycle process, stain is pumped into the chamber and incubated for 45 minutes, washed with PBS for 2.5 minutes and the slide was imaged. The next step in the cycle is the imaging, which has three parts (a) an auto focus step (b) an auto exposure step which updates the focus map and (c) an acquisition step to acquire the images. Following this is the dye inactivation step in which the imaging chamber is incubated with the dye inactivation solution for 3 minutes, followed by 2 minutes of washing. The fluorophore inactivated images were acquired using the exact same settings as the stained images. An overview of the process is shown in FIG. 8.

### Autofocus

Brenner scores are used as a metric for determining the relative focus of images. While this works well for ideal samples, it was found that it had two major limitations. Firstly, in field of views (FOVs) where there is minimal tissue, the standard Brenner scores is highly weighted towards any bright region. Secondly, the standard Brenner score takes the difference between pixels that are two pixels away from each other in the x axis, i.e. I_{x+2,y}−I_{x,y}. To practically improve the results for tissue-based applications, the Brenner score metric can be optimized by changing the number of pixels skipped. For a nuclei (DAPI) stain channel, it was found that a pixel skip size of 17 was optimal to maximize the Brenner score metric. To resolve the issue of minimal tissue in the FOV, a Brenner score was calculated constrained to only the tissue-positive region using a binary mask defined by the DAPI stain and using a standard StarDist network. Given that Brenner score is weighed by bright spots, it was observed that the presence of fluorophore aggregates often leads to erroneous focus maps. This was circumvented by using the log of the image when calculating the modified Brenner score.

For an image with n×n pixels with I_{x,y} being its intensity at coordinates (x,y) and a binary image mask where B_{x,y}=1 or 0 if nuclei are present at the coordinates (x,y), the focus scores were calculated as the following:

$$Standard Brenner Score : {\sum }_{y = 0}^{n} {\sum }_{x - 0}^{n - 2} {( {I}_{x , y} - {I}_{x + 2 , y} )}^{2}$$

$$Modified Brenner Score : {\sum }_{y = 0}^{n} {\sum }_{x = 0}^{n - 17} {( log \frac{{I}_{xy}}{{I}_{x + 17 , y}} )}^{2} * {B}_{x , y}$$

First, a 7-slice z stack with 2 μm step sizes in the DAPI channel is acquired. When the acquisition phase of the next cycle starts, the auto focus program uses the previous cycle's DAPI images to determine on a per tile basis the slice that has the highest modified Brenner score. It then calculates the AZ between the current focus maxima with previous cycle's maxima to center the stack around the current maxima and appends that value to the focus map. It was observed that the average drift going from cycle to the next is on the order of 2-4 μm. An overview of the autofocus process is shown in FIG. 9.

### Autoexposure

The objective of auto exposure is to achieve an optimal signal to noise (SNR) ratio for each fluorophore stain. Here, the percent dynamic range occupied (PDRO) which measures the percentage of the dynamic range in the intensity histogram normalized to the total dynamic range was used as an approximation for SNR. Autoexposure was determined as follows.
First, a binary mask was used to specifically identify the tissue regions. Signal intensity in these tissue-associated pixels can contain autofluorescence or actual stain which can be distinguished using Otsu's thresholding. From the subset of pixels which contain actual stain, the top and bottom 1% of the pixels by intensity are removed. These pixels are then used to determine the exposure time and the number of frames used for signal averaging.

$$1. \frac{P D R O}{Lowest Pixel intensity} * current exposure time = New Exposure Time$$

$$\begin{matrix}2. & \\ \frac{P D R O}{Lowest Pixel intensity} * Highest Pixel Intensity = Projected Intensity of Highest Pixel & \end{matrix}$$

$$2. \frac{P D R O}{Lowest Pixel intensity} * Highest Pixel Intensity =$$

$$\text{} Projected Intensity of Highest Pixel$$

$$3. round up ( \frac{0.85 * 16 bit depth well}{Projected Intensity of Highest Pixel} ) =$$

$$Number Frames to Average$$

$$4. \frac{New Exposure Time}{Number Frames to Average} = Average Adjusted Exposure Time$$

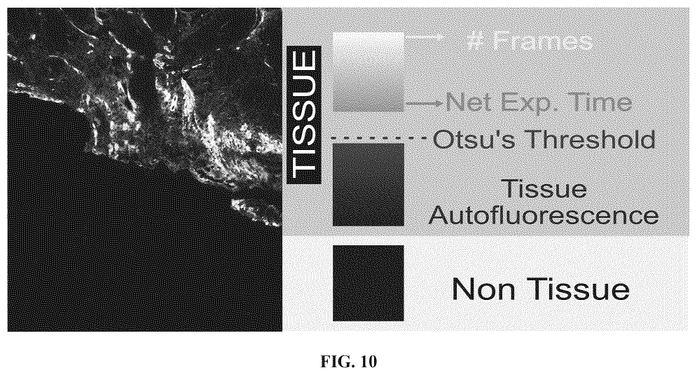

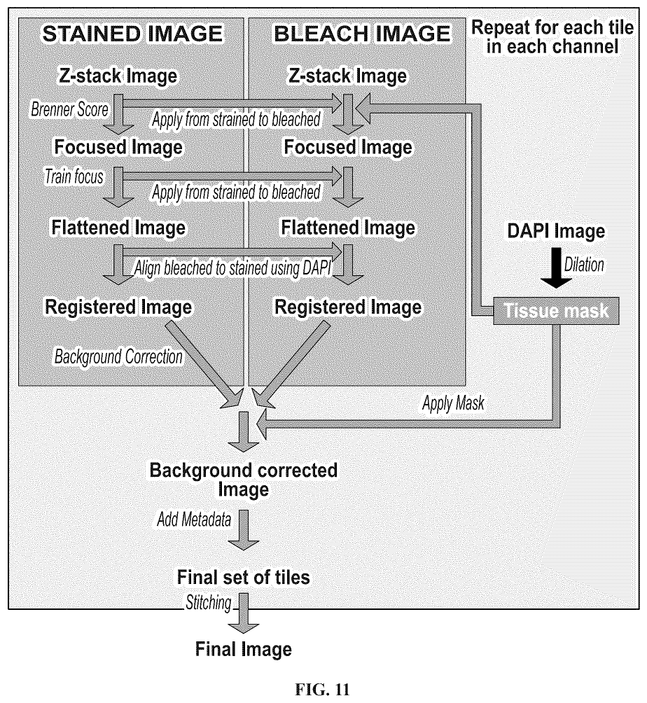

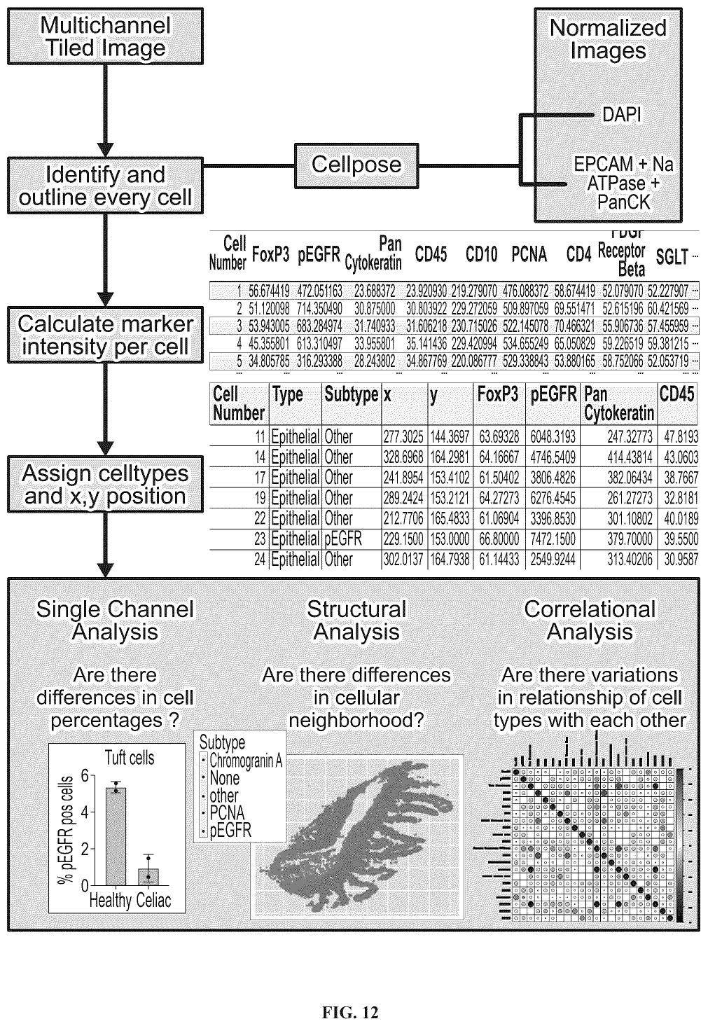

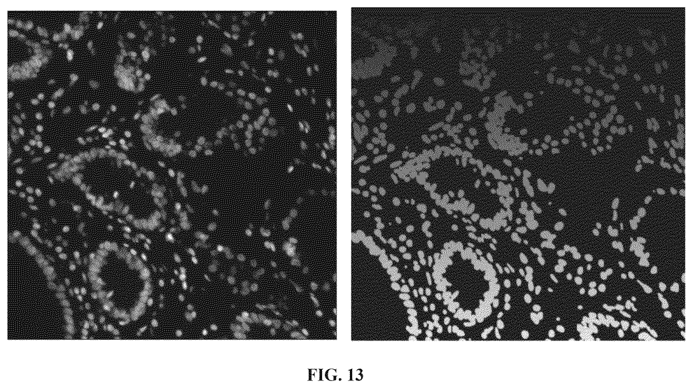

This method ensures that the dimmest pixels are adequately exposed, and the brightest pixels are not saturated. It was observed that while this method generally works well for most tissues, the presence of fluorophore aggregates tends to result in very small, calculated exposure times and underexposure of the actual stain. Similarly, very weak fluorophore staining tends to result in excessively high exposure times. To minimize the downstream effects on total imaging time that these scenarios can induce during automated operation of the SPECTRE-Plex system, hardcoded strict lower and upper limits were implemented for the exposure time for any given fluorophore. An overview of the autoexposure routine is shown in FIG. 10.

### Post-Processing Pipeline

Image processing of SPECTRE-plex datasets occurs in 3 major categories: i.) determination of the in-focus image, ii.) illumination normalization, and iii.) removal of background as outlined in FIG. 11. The standardized acquisition involves obtaining a z stack for each image area tile. This ensures that if the stain has a slightly different in focus location in z or if the DAPI offset is inaccurate for that specific tile, in focus information will still be captured. The modified Brenner score (see Supplementary 9) with a skip parameter of 10 instead of 17 is used to assess which z slice in the stack is in focus. The equivalent image in the dye-inactivated (‘bleach’) stack is obtained by using the same slice index as used for the stained z-stack. Next, the stained images that contain tissue regions are used to train a BaSiC flat field correction surface. This is applied to each stained and bleached tile. Since the bleached and stained images are gathered as part of different acquisition events, they may be slightly misaligned. Pystackreg is a python port of a turbo stack reg plugin from ImageJ and is used to register the bleached image to the stained image and output a bleached image following addition of a displacement vector. In practice this is generally only 1-2 pixels so no cropping is needed. Since the bleached image was acquired with the exact same setting as the stained, we subtracted the bleached image from the stained image. While this tends to leave some residual background, the majority is removed and additional processing such as a rolling ball background subtraction can be used if desired. After all tiles are processed, they are then packaged into tiff stacks and populated with metadata in such a way that they can be directly inputted into McMicro. The Ashlar module within the McMicro pipeline which registers cycles and stitches tiles together is used to generate a registered and stitched output file. Lastly, a binary tissue mask is generated from the DAPI channel and multiplied by the registered image set to remove any non-tissue signal to output a finalized dataset.

### Image Analysis

As the first step in image analysis, a tissue mask was created. In a twofold downsampled image, all the individual images in the multichannel image were normalized and added to create a composite stain image. This was then thresholded by Otsu's method to create a binary image which was then dilated. The largest contoured image with filled in holes was defined as the tissue mask. All subsequent image processing was conducted on images with the applied tissue mask. A schematic of the analysis pipeline is shown in FIG. 12.

Four channels (DAPI, Sodium Potassium ATPase, EpCAM, and Pan Cytokeratin) were normalized using Min Max Scaler in the sklearn python package. Sodium Potassium ATPase, EpCAM, and Pan Cytokeratin were then averaged together to create the final image for segmentation. CellPose (Ver 3.0.7) was then used for segmentation with the normalized cell boundary image and the normalized DAPI image being the “chan to segment” and “chan2” inputs respectively. The model used was “cyto3” with the default settings except for the “flow threshold” which was changed to 0.0. It was observed that in a random field of view shown in FIG. 13, this approach had an accuracy of 86% with a precision of 94% when compared to a manual analysis.

The mask along with the tiled images are then used for further analysis using a custom python code. Data structure was created to determine the mean intensity of each protein, along with the centroid coordinates for each cell in the image. The celltypes were then assigned using a set of criteria for each marker.

TABLE 2
Example cost of a SPECTRE-plex run

| | Cost | Size | Size | Amount | Cost |
|---|---|---|---|---|---|
| | of | of Vial | of Vial | per run | per run |
| Antibody | Vial | (mL) | (uL) | (uL) | ($) |
| Chromogranin A | 529 | 0.1 | 100 | 1 | 5.29 |
| sodium potassium | 610 | 0.1 | 100 | 2 | 12.2 |
| ATPase | | | | | |
| pEGFR | 565 | 0.1 | 100 | 5 | 28.25 |
| SGLT | 519 | 0.1 | 100 | 10 | 51.9 |
| Alexa Fluor ™ | 136.8 | 0.1 | 100 | 10 | 13.68 |
| 555 Antibody | | | | | |
| Labeling Kit | | | | | |
| Pan CytoKeratin | 455 | 0.5 | 500 | 5 | 4.55 |
| FoxP3 | 165 | 0.125 | 125 | 10 | 13.2 |
| smooth muscle | 590 | 0.1 | 100 | 2 | 11.8 |
| actin (SMA) | | | | | |
| CD45 | 435 | 0.1 | 100 | 10 | 43.5 |
| CD14 | 625 | 0.1 | 100 | 10 | 62.5 |
| CD4 | 249 | 0.5 | 500 | 10 | 4.98 |
| CD68 | 435 | 0.1 | 100 | 10 | 43.5 |
| MUC2 | 610 | 0.1 | 100 | 0.5 | 3.05 |
| Lactase | 449 | 0.1 | 100 | 2 | 8.98 |
| CD3D | 600 | 0.1 | 100 | 5 | 30 |
| gamma actin | 350 | 1 | 1000 | 5 | 1.75 |
| CD10 | 350 | 0.1 | 100 | 10 | 35 |
| PDGF Receptor | 217.5 | 0.05 | 50 | 5 | 21.75 |
| Beta | | | | | |
| CD31 | 650 | 0.1 | 100 | 10 | 65 |
| PCNA | 350 | 1 | 1000 | 25 | 8.75 |
| EpCAM | 635 | 0.1 | 100 | 2 | 12.7 |
| Lysozyme | 499 | 0.1 | 100 | 5 | 24.95 |
| DAKO Diluent | 320 | 125 | 125000 | 2800 | 7.168 |
| Hoescht | 135 | 10 | 10000 | 0.2 | 0.0027 |
| PBS | | | | | 15.17 |
| Total Cost | | | | | 529.62 |

### Human Tissue Sections

De-identified formalin-fixed paraffin embedded duodenal tissue sections for multiplex staining were obtained under Boston Children's Hospital IRB protocol #P00046566. Celiac disease (active disease, serologically positive) and age-matched healthy control sections were obtained from <12 yo subjects.

### OTHER EMBODIMENTS

From the foregoing description, it will be apparent that variations and modifications may be made to the disclosure described herein to adapt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An optical system for multiplex image acquisition, the system comprising
a) a microscope comprising
at least one light source configured to emit light towards a sample present in a chamber disposed within a chamber body;
an upright water dipping objective lens or water immersion lens configured to receive the light;
and a digital camera, a photodiode, or an optical detector configured to extract light captured by the objective lens, thereby taking an image of a sample;
b) a microfluidic chamber comprising an optical window positioned between the sample and the lens of the objective, wherein the viewing window has approximately the same refractive index as the solution in which the water-dipping objective is immersed and the solution present in the microfluidic chamber in which the sample is immersed, wherein the microfluidic chamber comprises at least one inlet in fluid communication with the chamber, and at least one outlet in fluid communication with the chamber, wherein the flow of liquid through the microfluidic chamber is controlled by a controller operably connected to the microfluidic chamber;
c) a motorized stage in which the microfluidic chamber is disposed and at least one controller operably connected to the motorized stage, wherein the controller is configured to position the stage with submicron precision in X and Y axes, wherein the position of the stage is controlled by a controller operably connected to the motorized stage.

2. The system of claim 1, wherein the microscope is an epifluorescence, confocal, or light sheet microscope;
wherein the microfluidic chamber is a plurality of microfluidic chambers;
wherein the viewing window comprises fluorinated ethylene propylene (FEP) film; and
wherein the microfluidic chamber is disposed within a motorized stage, the motorized stage comprising
a metal plate configured to hold a liquid, the plate comprising at least two magnets;
a chamber body comprising Fe₂O₃ embedded Polydimethylsiloxane (PDMS) configured to hold a fluorinated ethylene propylene (FEP) film;
a sample chamber comprising an optical window, wherein the sample chamber is configured to accept a biological sample, the sample chamber further comprising a microfluidic system comprising at least one inlet in fluid communication with the sample chamber and at least one outlet in fluid communication with the sample chamber;
a planar surface comprising anti-reflective material positioned below the sample chamber;
a planar surface comprising a thermosetting resin that provides insulation, thermal conductivity, and/or mechanical support to the sample chamber.

3. A microfluidic system comprising:
a microfluidic chamber comprising:
a chamber body, and
a viewing window comprising a fluorinated ethylene propylene (FEP) film.

4. The system of claim 1, further comprising a magnet positioned below the chamber body.

5. The system of claim 1, wherein the chamber body is made from a Polydimethylsiloxane (PDMS) material and embedded with iron oxide dust.

6. The system of claim 1, wherein the viewing window is installed in the chamber body while the PDMS is in a curing state, the viewing window further comprising a FluoroEtch chemical material in the FEP film to increase bonding between the FEP film and the PDMS material of the chamber body.

7. The system of claim 1, wherein the magnet is configured to enhance bond strength in the PDMS material, the magnet causing a magnetic force acting on the iron oxide dust of the PDMS material.

8. A microfluidic chamber comprising a viewing window comprising a fluorinated ethylene propylene (FEP) film, at least one inlet, and at least one outlet, the inlet and outlet in fluid communication with the microfluidic chamber, wherein the flow of liquid through the microfluidic chamber is controlled by a controller operably connected to the microfluidic chamber.

9. A microfluidic system comprising:
a widefield upright microscope having a high-resolution XY stage with a fluid-immersion chamber and a slide surface, the fluid-immersion chamber having a magnet-embedded slide;
a microfluidic chamber positioned on the magnet-embedded slide and having
a chamber body made from a Polydimethylsiloxane (PDMS) material and embedded with iron oxide dust, and
a viewing window made from fluorinated ethylene propylene (FEP) film, the viewing window further including a FluoroEtch chemical material in the FEP film to increase bonding between the FEP film and the PDMS material of the chamber body; and
a magnet positioned below the slide surface, the magnet causing a magnetic force acting on the iron oxide dust of the PDMS material.

10. The microfluidic system of claim 9, wherein the widefield upright microscope includes a high numerical aperture (NA) water dipping objective; and/or wherein the slide surface has a heating element embedded thereof.

11. The microfluidic system of claim 9, further comprising a micro-peristaltic pump configured to control, via a multipinch valve, flow of fluorophores within the microfluidic chamber.

12. The microfluidic system of claim 9, wherein the FEP film has a refractive index that is the same as the refractive index of the water.

13. A microfluidic system comprising:
a widefield upright microscope having a high-resolution XY stage with a slide surface;
a microfluidic chamber positioned on the slide surface and having
a chamber body made from a Polydimethylsiloxane (PDMS) material and embedded with iron oxide dust, and
a viewing window made from fluorinated ethylene propylene (FEP) film, the viewing window further including a FluoroEtch chemical material in the FEP film;
a micro-peristaltic pump configured to control flow of fluorophores within the microfluidic chamber;
a magnet positioned below the slide surface, the magnet causing a magnetic force acting on the iron oxide dust of the PDMS material,
and a composition selected from the group consisting of buffer, a capture molecule conjugated to a fluorophore, and meta-chloroperoxybenzoic acid.

14. A method of inactivating fluorescence from a fluorophore, the method comprising: contacting a fluorophore with a peroxy acid.

15. A method of inactivating a fluorophore bound to a biological sample, the method comprising:
a) contacting a biological sample with a capture molecule conjugated to a fluorphore, wherein the capture molecule specifically binds a target present in the biological sample; and
b) contacting the sample with a peroxy acid, thereby inactivating the fluorophore.

16. The method of claim 14, wherein the peroxy acid is present in a basic solution.

17. A multiplex method for detecting two or more target molecules in a sample, the method comprises:
a) contacting a biological sample with a capture molecule conjugated to a first fluorophore, wherein the capture molecule specifically binds a target molecule within the sample;
b) exciting the first fluorophore with an appropriate wavelength of light and detecting the fluorescence;
c) inactivating the fluorescence by contacting the biological sample with a basic composition comprising a peroxy acid; and
d) repeating steps a-c with one or more capture molecules, each conjugated to a fluorophore.

18. The method of claim 17, wherein the target molecule of the first capture molecule is the same or different from the target bound by a subsequent capture molecule.

19. The method of claim 17, wherein fluorophore conjugated to the first capture molecule is the same or different from the fluorphore conjugated to a subsequent capture molecule.

20. The method of claim 17, wherein the peroxy acid is selected from the group consisting of peracetic acid; m-CPBA; magnesium monoperphthalate; Payne's reagent; trifluoroperacetic acid; 2,4-dinitroperbenzoic acid; Caro's acid; and potassium caroate.
